SEQUENCE LISTING (8 sequences)

SEQ ID NO: 1 - DNA, 1587 bp, Mouse
tatccgccagaagagagaccagccgagaagaggtgagcacaactaagcaatacctccttcacttctctgctctctgctggggacagaggaaaggcttataactctgcaagtgttgtctgaagctagcccataccaggcagtgtagtgacacaggacccctcacttaagcagtacaaggtgagacagagactttcaacacccagcctggaggaataatggtcttcattctcttgagtccaaccagggtttgactgactaaatgaggcactggccatggctcatctctttaaagagaaaagtaactgttatttctgcttccggtgtctggaaagccctgtgtacttgaactgtggatacatctgctgcctcaagtgccttgactcactagagaaaagtcctgaaggggacggtgtactgtgccccacttgctctgttgtctctttgaaagaagacatcatacatgctaaacagttaggggccctggttaccaagatcaaaaacctagagccacagctgaattttattctgacaatggaccaaggtatgaagatatttcaagtaaccatgaccttggatgtggacacagcccagaaccacctcatcatctctgatgacctgctgagtgtctactatacgcctcagaagcaagcccgaaagaaatgtgcagaaagattccatccctccccttgtgtcctgggctcttcccggttcacttcaggccgccattactgggaggtagtggtgggaaccagcaaagaatgggatataggcatttgcaaagagtccattaatcgaaaaaaggctattcatttgtctgaaaaaaatggcttctggaccgtgggtgtgagggcaaaaaaggtctattctgccagcactgaccccttgactgtgctgcgtgtgaaccctcggctacgtagagtgggcattttccttgacatgctagagaaaagtgtttctttctgggaccttagcgatggctcccatatctatacattccttgaaattcctgacacggatccatttcgcccattcttttctccagcaagttcctatccagatggtgatcaagaacaagtcctgagtatctgtcctgtgacaaatccaggcattttcggaattccagttaacccccaataaggaaaataacccttgggtatgaaagctgctatgacagtcgctgagtactcataacctagctaagaatttttgagtctggtacccatggagcacaaaagcattacagaaatgtatggagggcctcataaatactaactagttacaggaaatgccatttgatttggtttggtttggtttggttttgggtttttttttttaattttttttacactccatattttattttccctcccacccatgccccgtcctcaagagactagaggccctagggagtttagaggaaatagcattttaatataaagtattatcatgattgttttctttggaatttatgtttatatttctgttcccattcatagattgtgaaattttactgtgaatgctttcctttttatttgctataatattagtgtatgatgttgtggaataaataaactaaattaaaatttt

SEQ ID NO: 2 - DNA, 816 bp, Human
atggctgaacactttaaacaagcaagcagttgtcctatctgcctggattatcttgaaaaccccacgcacctgaaatgtggatacatctgttgcctccgatgcatgaactcactgcgaaaggggcccgatgggaagggggtgctgtgccctttctgccctgtggtctctcagaaaaatgacatcaggcccgctgcccagctgggggcgctggtgtccaagatcaaggaactagagcccaaggtgagagctgttctgcagatgaatccaaggatgagaaagttccaagtggatatgaccttggatgtggacacagccaacaacgatctcatcgtttctgaagacctgaggcgtgtccgatgtgggaatttcagacagaataggaaggagcaagctgagaggttcgacactgccctgtgcgtcctgggcacccctcgcttcacttccggccgccattactgggaggtgggcgtgggcaccagccaagtgtgggatgtgggcgtgtgcaaggaatctgtgaaccgacaggggaacgttgtactctcttcagaactcggcttctggactgtgggtttgagacaaggacagatctactttgccagcactaagcctgtgacgggtctctgggtgagctcaggtctacaccgagtggggatttacctggatataaaaacgagggccatttccttctataatgtcagtgataggtcacatatcttcacattcacgaaaatttctgctactgagccactgcgcccatgttttgctcatgcagatacaagtcgtgatgatcacggatacttgagtgtgtgtgtgtaa

SEQ ID NO: 3 - protein, 287 aa, Mouse
MAHLFKEKSNCYFCFRCLESPVYLNCGYICCLKCLDSLEKSPEGDGVLCPTCSVVSLKEDIIHAKQLGALVTKIKNLEPQLNFILTMDQGMKIFQVTMTLDVDTAQNHLIISDDLLSVYYTPQKQARKKCAERFHPSPCVLGSSRFTSGRHYWEVVVGTSKEWDIGICKESINRKKAIHLSEKNGFWTVGVRAKKVYSASTDPLTVLRVNPRLRRVGIFLDMLEKSVSFWDLSDGSHIYTFLEIPDTDPFRPFFSPASSYPDGDQEQVLSICPVTNPGIFGIPVNPQ

SEQ ID NO: 4 - protein, 271 aa, Human
MAEHFKQASSCPICLDYLENPTHLKCGYICCLRCMNSLRKGPDGKGVLCPFCPVVSQKNDIRPAAQLGALVSKIKELEPKVRAVLQMNPRMRKFQVDMTLDVDTANNDLIVSEDLRRVRCGNFRQNRKEQAERFDTALCVLGTPRFTSGRHYWEVGVGTSQVWDVGVCKESVNRQGNVVLSSELGFWTVGLRQGQIYFASTKPVTGLWVSSGLHRVGIYLDIKTRAISFYNVSDRSHIFTFTKISATEPLRPCFAHADTSRDDHGYLSVCV

SEQ ID NO: 5 - protein, 285 aa, Human
MAEHFKQASSCPICLDYLENPTHLKCGYICCLRCMNSLRKGPDGKGVLCPFCPVVSQKNDIRPAAQLGALVSKIKELEPKVRAVLQMNPRMRKFQVDMTLDVDTANNDLIVSEDLRRVRCGNFRQNRKEQAERFDTALCVLGTPRFTSGRHYWEVGVGTSQVWDVGVCKESVNRQGNVVLSSELGFWTVGLRQGQIYFASTKPVTGLWVSSGLHRVGIYLDIKTRAISFYNVSDRSHIFTFTKISATEPLRPCFAHADTSRDDHGYLSVCVINNGIASSPIYPGQ

SEQ ID NO: 6 - DNA, 855 bp, Human
atggctgaacactttaaacaagcaagcagttgtcctatctgcctggattatcttgaaaaccccacgcacctgaaatgtggatacatctgttgcctccgatgcatgaactcactgcgaaaggggcccgatgggaagggggtgctgtgccctttctgccctgtggtctctcagaaaaatgacatcaggcccgctgcccagctgggggcgctggtgtccaagatcaaggaactagagcccaaggtgagagctgttctgcagatgaatccaaggatgagaaagttccaagtggatatgaccttggatgtggacacagccaacaacgatctcatcgtttctgaagacctgaggcgtgtccgatgtgggaatttcagacagaataggaaggagcaagctgagaggttcgacactgccctgtgcgtcctgggcacccctcgcttcacttccggccgccattactgggaggtgggcgtgggcaccagccaagtgtgggatgtgggcgtgtgcaaggaatctgtgaaccgacaggggaacgttgtactctcttcagaactcggcttctggactgtgggtttgagacaaggacagatctactttgccagcactaagcctgtgacgggtctctgggtgagctcaggtctacaccgagtggggatttacctggatataaaaacgagggccatttccttctataatgtcagtgataggtcacatatcttcacattcacgaaaatttctgctactgagccactgcgcccatgttttgctcatgcagatacaagtcgtgatgatcacggatacttgagtgtgtgtgtaattaataatggcattgccagttccccaatttatcctgggcaa

SEQ ID NO: 7 - DNA, 20 bp, Artificial Sequence
  Primer
gggtgggagggaaaataaaa

SEQ ID NO: 8 - DNA, 20 bp, Artificial Sequence
  Primer
ggtacccatggagcacaaag

(19) United States
(12) Patent Application Publication
(10) Pub. No.: US 2003/0144205 A1
(43) Pub. Date: Jul. 31, 2003

(54) Novel germ cell-specific contraceptive target

(76) Inventors: Martin M. Matzuk, Pearland, TX (US); Aleksander Rajkovic, Houston, TX (US); Nobuhiro Suzumori, Showa-Ku (JP); Kathleen H. Burns, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/325,273

(22) Filed: Dec. 20, 2002

Related U.S. Application Data
(60) Provisional application No. 60/345,164, filed on Dec. 21, 2001.
(60) Provisional application No. 60/398,407, filed on Jul. 25, 2002.

(51) Int. Cl. A61K 38/16; C07H 21/04; C12N 9/64; C12P 21/02; C12N 5/06; C07K 16/40

(52) U.S. Cl. 514/12; 530/388.26; 435/69.1; 435/226; 435/320.1; 435/325; 536/23.2

(57) ABSTRACT

The present invention relates to compositions and methods for modulating conception in animals. More particularly, the composition modulates protein degradation during gametogenesis and early development.

This application claims priority to U.S. Provisional Application serial Nos. 60/345,164 filed on Dec. 21, 2001 and 60/398,407 filed on Jul. 25, 2002.

This invention was made with government support under NIH-NICHD Grant Nos. HD33438, HD37231, and HD00849 awarded by the National Institutes of Health. The United States Government may have certain rights in the invention.

### BACKGROUND

I. Field of Invention

The present invention relates to the field of medicine. More particularly, it relates to pharmaceutical compositions and methods for modulating conception in animals.

II. Related Art

Ubiquitin-mediated protein degradation pathways play important regulatory roles in diverse cellular processes, modulating cell cycle progression, intracellular signaling cascades, transcription, and apoptosis (Freemont, 2000; Joazeiro and Weissman, 2000). Degradation enzymes target proteins for proteolysis by the covalent addition of ubiquitin polymers. There are three major classes of enzymes that act sequentially to ubiquitinate a target protein. E1 (ubiquitin-activating) and E2 (ubiquitin-conjugating) enzymes prepare the polyubiquitin. RING finger proteins function as E3 ubiquitin-protein ligases, transferring ubiquitin polymers from E2 ubiquitin-conjugating enzymes to recipient proteins that are thus marked for proteolysis. Few E1 enzymes, several E2 enzymes, and hundreds of E3 enzymes, which determine the specificity of the system (i.e., the proteins to be degraded) have been identified. The RING finger motif, which is characteristic of E3 ubiquitin-protein ligases, is defined by a series of conserved cysteine and histidine residues. These motifs are critical to E3 enzyme functions. Mutations within the RING finger-coding portion of the X-linked MID1 gene lead to Opitz G/BBB syndrome, a defect in midline development (Quaderi et al., 1997). Mutations affecting the BRCA1 RING finger domain lead to misregulated cell division and familial carcinomas (Thai et al., 1998; Wu et al., 1996). Mutations in the E3 RING finger protein Parkin result in autosomal recessive juvenile parkinsonism. The effects of these mutations in vivo demonstrate the importance of these E3 proteins.

Although it is clear that protein turnover is exquisitely regulated during gametogenesis and early embryonic development, to date few components of a germ cell-specific ubiquitination pathway have been identified. Thus, the inventors of the present invention have identified the first germ cell-specific E3 protein. It is envisioned that modulation of this protein plays a role in contraception and fertility.

### BRIEF SUMMARY OF THE INVENTION

The present invention is drawn to a novel polynucleotide, polypeptide and variants thereof. Compositions of the present invention are used to modulate protein degradation. It is envisioned that the novel polynucleotide or polypeptide mediate protein degradation pathways important for gametogenesis or early embryonic development.

One embodiment of the present invention is an isolated polynucleotide sequence encoding a polypeptide comprising an amino acid sequence of SEQ.ID.NO: 3, SEQ.ID.NO: 4 or SEQ.ID.NO: 5. Yet further, a specific embodiment is an isolated polypeptide comprising an amino acid sequence of SEQ.ID.NO: 3, SEQ.ID.NO: 4, or SEQ.ID.NO: 5. Further embodiments can also include a monoclonal antibody or polyclonal antisera that bind(s) immunologically to a polypeptide comprising SEQ.ID.NO: 3, SEQ.ID.NO: 4, or SEQ.ID.NO: 5 or an antigenic fragment thereof.

A further embodiment is an expression vector comprising a polynucleotide sequence encoding a polypeptide having an amino acid sequence of SEQ.ID.NO: 3, SEQ.ID.NO: 4, or SEQ.ID.NO: 5 wherein said polynucleotide is under control of a promoter. Specifically, the polynucleotide sequence comprises SEQ.ID.NO: 1, SEQ.ID.NO: 2, or SEQ.ID.NO: 6. Yet further, a specific embodiment comprises the host cell transformed with the expression vector described herein.

Another embodiment is an isolated polynucleotide sequence comprising a nucleic acid sequence of SEQ.ID.NO: 1, SEQ.ID.NO: 2, or SEQ.ID.NO: 6. Yet further, a specific embodiment is an antisense molecule comprising the complement of the polynucleotide of SEQ.ID.NO: 1, SEQ.ID.NO: 2, or SEQ.ID.NO: 6 or a functional equivalent thereof.

Another embodiment of present invention further comprises a method for producing a polypeptide comprising the steps of: culturing a host cell under conditions suitable for the expression of said polypeptide; and recovering the polypeptide from the host cell culture.

Yet further, another embodiment is a pharmaceutical composition comprising a modulator of RFPL4 expression and/or activity dispersed in a pharmaceutically acceptable carrier. The modulator suppresses or enhances transcription of an RFPL4 gene or inhibits or stimulates RFPL4 activity. The modulator can also suppress or enhance translation of an RFPL4 transcript or alter RNA stability by increasing or decreasing RNA degradation. More particularly, the modulator is a polypeptide, a polynucleotide sequence (DNA or RNA) or a small molecule. In a specific embodiment, the pharmaceutical -composition comprises an expression vector, wherein the expression vector comprises a promoter and the polynucleotide sequence, operatively linked.

Another embodiment is a method of identifying compounds that modulate the activity of RFPL4 comprising the steps of: obtaining an isolated RFPL4 polypeptide or functional equivalent thereof; admixing the RFPL4 polypeptide or functional equivalent thereof with a candidate compound; and measuring an effect of the candidate compound on the activity of RFPL4. The effect is a decrease and/or increase in protein degradation.

Still further, another embodiment is a method of screening for a modulator of RFPL4 activity comprising the steps of: providing a cell expressing an RFPL4 polypeptide; contacting the cell with a candidate modulator; measuring RFPL4 expression; and comparing the RFPL4 expression in the presence of the candidate modulator with the expression of RFPL4 expression in the absence of the candidate modulator; wherein a difference in the expression of RFPL4 in the presence of the candidate modulator, as compared with the expression of RFPL4 in the absence of the candidate modulator, identifies the candidate modulator as a modulator of RFPL4 expression.

A specific embodiment is a method of producing a modulator of RFPL4 activity comprising the steps of: providing a cell expressing an RFPL4 polypeptide; contacting the cell with a candidate modulator; measuring RFPL4 expression; comparing the RFPL4 expression in the presence of the candidate modulator with the expression of RFPL4 expression in the absence of the candidate modulator; wherein a difference in the expression of RFP4 in the presence of the candidate modulator, as compared with the expression of RFPL4 in the absence of the candidate modulator, identifies the candidate modulator as a modulator of RFPL4 expression; and producing the modulator.

Another specific embodiment is a method of modulating protein degradation in a germ cell or early embryo of an animal comprising the step of administering to the animal an inhibitor of RFPL4 activity. The germ cell is an oocyte or egg and/or a spermatogonium, spermatocyte, spermatid or spermatazoon. The inhibitor suppresses transcription of an RFPL4 gene, suppresses translation of an RFPL4 transcript or increases RNA degradation of an RFPL4 transcript. More particularly, the inhibitor is a polypeptide or a polynucleotide (DNA or RNA). Yet further, the RNA is antisense RFPL4 RNA or an RNA interference of RFPL4 RNA. In specific embodiments, the inhibitor is an expression vector, wherein the expression vector comprises a promoter and the polynucleotide sequence, operatively linked. The vector is a bacterial, viral or mammalian vector.

A further embodiment is a method of contraception comprising administering to an animal an effective amount of an inhibitor of RFPL4 activity dispersed in a pharmacologically acceptable carrier, wherein the amount is capable of decreasing conception. The animal is female or male. The inhibitor suppresses transcription of an RFPL4 gene, suppresses translation of an RFPL4 transcript or increases RNA degradation of an RFPL4 transcript. More particularly, the inhibitor is a polypeptide, a polynucleotide (DNA or RNA), or a small molecule. Yet further, the RNA is antisense RFPL4 RNA or an RNA interference of RFPL4 RNA. In specific embodiments, the inhibitor is an expression vector, wherein the expression vector comprises a promoter and the polynucleotide sequence, operatively linked. The vector is a bacterial, viral or mammalian vector.

Still further, another embodiment is a method of contraception comprising administering to an animal an effective amount of a stimulator of RFPL4 activity dispersed in a pharmacologically acceptable carrier, wherein said amount is capable of decreasing conception. The animal is female or male. The stimulator enhances transcription of an RFPL4 gene, enhances translation of an RFPL4 transcript or decreases RNA degradation of an RFPL4 transcript. The stimulator is a polypeptide, a polynucleotide (DNA or RNA), or a small molecule. Yet further, the RNA is antisense RFPL4 RNA or an RNA interference of RFPL4 RNA. In specific embodiments, the stimulator is an expression vector, wherein the expression vector comprises a promoter and the polynucleotide sequence, operatively linked. The vector is a bacterial, viral or mammalian vector.

Another embodiment is a method of modulating protein degradation in a germ cell or early embryo of an animal comprising the step of administering to the animal a stimulator of RFPL4 activity. The germ cell is an oocyte or egg and/or a spermatogonium, spennatocyte, spermatid or spermatazoon. The stimulator enhances transcription of an RFPL4 gene, enhances translation of an RFPL4 transcript or decreases RNA degradation of an RFPL4 transcript. The stimulator is a polypeptide, a polynucleotide sequence (DNA or RNA), or a small molecule. Yet further, the RNA is antisense RFPL4 RNA or an RNA interference of RFPL4 RNA. In specific embodiments, the stimulator is an expression vector, wherein the expression vector comprises a promoter and the polynucleotide sequence, operatively linked. The vector is a bacterial, viral or mammalian vector.

Still further, another embodiment is a method of enhancing fertility comprising administering to an animal an effective amount of an inhibitor or a stimulator of RFPL4 activity dispersed in a pharmacologically acceptable carrier, wherein the amount is capable of decreasing conception.

Yet further, another embodiment is a method of diagnosing infertility comprising identifying a mutation in an RFPL4 polypeptide or polynucleotide. The method comprises identifying a mutation in an RFPL4 polypeptide or in an RFPL4 polynucleotide, for example MRNA or DNA.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended sentences. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying figures.

FIG. 1 shows Rfpl4 cDNA and its translation. The Rfpl4 CDNA is 1581 nucleotides long, and encodes a 287 amino acid protein. The amino terminus is cysteine-rich; cysteine residues and the tyrosine that are part of the RING finger-like domain are demarcated in bold font and tyrosine is boxed. The B30.2 domain is underlined with a solid line. Amino acid numbering is displayed on the left side of the figure, and nucleotide numbering is displayed on the right.

FIG. 2A, FIG. 2B, FIG. 2C, FIG. 2D and FIG. 2E show alignment of RING finger-like domains. FIG. 2A shows the mouse RFPL4 (mRFPL4) and human RFPL4 (hRFPL4) RING finger-like domains were aligned with human RFPL1 (hRFPL1), RFPL2 (hRFPL2), RFPL3 (hRFPL3), as well as mouse ret finger protein (mRFP) which contains a “classical” RING domain. The conserved cysteines of the C3NC4 RING finger-like domains are boxed. An arrow indicates the position of the conserved histidine amino encountered in mRFP but substituted in the RFPL1, RFPL2, and RFPL3 with cysteine and substituted in the RFPL4 with tyrosine. FIG. 2B shows the mouse RFPL4 (mRFPL4) and human RFPL4 (hRFPL4) alignment. Human RFPL4 is 271 amino acids long based on virtual translation of genomic database sequences and is highly homologous to the mouse RFLP4. The conserved cysteines of the RING finger-like domain are indicated with short arrows, and the conserved tyrosine is indicated with asterisk. FIG. 2C shows 816 nucleotides of the human RFPL4 gene sequences that encode the deduced 271 amino acid human RFPL4 protein. FIG. 2D shows 833 nucleotides of a variant human RFPL4 gene sequence that encodes a variant human RFPL4 protein. FIG. 2E shows a 285 amino acid variant human RFPL4 gene sequence that was deduced from the variant human RFPL4 gene sequences. The human RFPL4 cDNA is predicted to be at least 816 or 833 nucleotides long and also include 5′ and 3′ untranslated regions. The human RFPL4 MRNA and the human RFPL4 protein are predicted to be gonad and early embryo-specific in their expressions and functions based on experimental findings, database searches, and experimental analysis of the functions of mouse RFPL4.

FIG. 3A and FIG. 3B show Rfpl4 expression in adult gonads. FIG. 3A shows RT-PCR analysis of Rfpl4 transcripts. FIG. 3B shows tissue-specific expression of Rfpl4 transcripts.

FIG. 4A, FIG. 4B, FIG. 4C, FIG. 4D, FIG. 4E and FIG. 4F show in situ hybridization of Rfpl4 in adult gonads. In situ hybridization of 12 week-old wild-type mouse ovaries and testes using an antisense riboprobe generated from the Rfpl4 CDNA fragment. FIGS. 4A, 4C, and 4E show a brightfield view of Hematoxylin-stained mouse ovaries and testes. FIGS. 4B, 4D, and 4F show corresponding darkfield views demonstrating that Rfpl4 transcripts are expressed in oocytes and spermatids. The outer ring observed in darkfield views is confined to oocytes and does not involve granulosa cells.

FIG. 5A and FIG. 5B show genomic organization of Rfpl4. FIG. 5A is a schematic representation of the Rfpl4 locus. Initiator methionine (AUG) and stop codon (TAA) are shown with their respective nucleotide positions in the cDNA. The coding region is demarcated by filled boxes and encodes 287 amino acids. FIG. 5B shows the three exons corresponding to 258, 296 and 1029 bp that are separated by two introns. The sequences of the exon-intron junctions are shown.

FIG. 6 shows a Western blot analysis of RFPL4 expression. Anti-RFPL4 goat polyclonal antibodies detected RFPL4 protein in lysates from wild-type (Wt) and Gdf9−/−ovary samples and early embryos, but not from heart (He), liver (Li), spleen (Sp), or testes (Te). RFPL4 protein is present in unfertilized GV stage oocytes (Oo), and 2-cell embryos, but not in 8-cell embryos. Actin in the tissue lysates was shown as a control for protein loading.

FIG. 7A, FIG. 7B, and FIG. 7C, show immunohistochemical analysis of RFPL4 in mouse ovary. FIG. 7D, FIG. 7E, FIG. 7F, FIG. 7G, and FIG. 7H show immunofluorescence analysis to detect RFPL4 in oocytes and early embryos. Seven week old wild-type (FIG. 7A, FIG. 7B) and Gdf9 knockout ovary (FIG. 7C) photographed under low (FIG. 7A, FIG. 7B) or high (FIG. 7C) magnification. In the adult wild-type ovary (FIG. 7A, FIG. 7B), RFPL4 immunoreactivity was detected in the cytoplasm of oocytes in the primary (1F), secondary (2F), and antral follicles (AF). The cytoplasm of oocytes in antral follicles (AF) of the wild-type mouse ovary are heavily stained (FIG. 7B). Folliculogenesis is blocked at primary follicle stage in Gdf9 knockout mice (Dong et al., 1996). In the Gdf9 knockout mouse ovary, RFPL4 immunoreactivity was detected in the cytoplasm of oocytes in the primary follicles (FIG. 7C). RFPL4 protein was detected by immunofluorescence in oocytes preceding the resumption of meiosis (FIG. 7D). It was predominantly cytoplasmic, and there was relative exclusion of RFPL4 from the nucleolus. RFPL4 was rapidly degraded in early preimplantation embryos between the 2-cell (FIG. 7E) and 8-cell stage (FIG. 7F). FIG. 7G shows two unfertilized oocytes arrested in metaphase II next to a blastocyst with no detectable RFPL4 protein. Preimmune goat serum was used as a negative control; a GV stage oocyte was shown before resumption of meiosis surrounded by granulosa cells (FIG. 7H).

FIG. 8 shows quantitative analysis of RFPL4 immunofluorescence in oocytes and early embryos. High levels of RFPL4 were detected in GV stage oocytes (GV) and metaphase II oocytes (MII). RFPL4 signal was diminished in 2-cell (2c) and 4-cell (4c) embryos, and was not detected in 8-cell morula. Average intensities after 0.5 s exposures were given with prebleed intensities subtracted.

FIG. 9A and FIG. 9B show truncation constructs of RFPL4 and cyclin B 1. FIG. 9A shows a schematic representation of RFPL4, and ΔC79, ΔN86, ΔN79ΔC155, ΔC155, and ΔN79 derivatives. The RING finger-like region and B30.2 domain are shown as gray and black boxes, respectively. FIG. 9B shows a schematic representation of cyclin B1, and ΔC198, and ΔN251 derivatives. The destruction box (D-box) and cyclin box are shown as gray and black boxes, respectively.

FIG. 10A, FIG. 10B and FIG. 10C show the strength of protein-protein interactions assessed by cotransformant and yeast mating fluorescence assays. FIG. 10A shows the cotransformant assay. FIG. 10B and FIG. 10C show the yeast mating assay.

FIG. 11A, FIG 11B, FIG. 11C and FIG. 11S show immunoprecipitation (IP) with anti-FLAG antibodies. FIG. 11A shows that in Western blotting, MYC-tagged proteins were detected with anti-MYC antibodies. FIG. 11B shows that FLAG-tagged RFPL4 was detected with anti-FLAG antibodies. The MYC-tagged HR6A, cyclin B1, and PSMB1 all were detected in the immunoprecipitate with FLAG-tagged RFPL4. FIG. 11C shows that MYC-tagged HR6A and RFPL4 were immunoprecipitated in association with FLAG-tagged cyclin B1. FIG. 11D shows that FLAG-tagged cyclin B1 was detected with anti-FLAG antibodies (1:1000 dilution), and MYC-tagged constructs were detected with anti-MYC antibodies (1:1000 dilution).

FIG. 12 shows a schematic representation of RFPL4 in complex with MPF and factors of the ubiquitin-mediated proteosomal degradation pathway.

FIG. 13A and FIG. 13B show cell-free transcription/translation of Rfpl4, HR6A, cyclin B1 (N, N-terminal; C, C-terminal), PSMB1 cDNAs, followed by co-immunoprecipitation and SDS-PAGE. Autoradiograph of [³⁵S]Met-labeled proteins from cell-free in vitro transcription/translation and co-immunoprecipitation by anti-HA polyclonal antibody (FIG. 13A) or anti-MYC monoclonal antibody (FIG. 13B). The position of molecular mass standards in kDa is shown at left.

FIG. 14A and FIG. 14B show cell-free transcription/translation of Rfpl4 and Zar1 cDNAs, followed by co-immunoprecipitation and SDS-PAGE. FIG. 14A shows in vitro transcription/translation and co-immunoprecipitation by anti-HA polyclonal antibody. FIG. 14B shows in vitro transcription/translation and co-immunoprecipitation by anti-MYC polyclonal antibody.

FIG. 15 shows a gene targeting construct to produce Rfpl4 knockout using ES cell technology. The construct deletes exon 2 encoding the initiation methionine to produce the null allele.

### DETAILED DESCRIPTION OF THE INVENTION

It is readily apparent to one skilled in the art that various embodiments and modifications can be made to the invention disclosed in this Application without departing from the scope and spirit of the invention.

I. Definitions

As used herein, the use of the word “a” or “an” when used in conjunction with the term “comprising” in the sentences and/or the specification may mean “one,” but it is also consistent with the meaning of “one or more,” “at least one,” and “one or more than one.”

As used herein, the term “animal” refers to a mammal, such as human, non-human primates, horse, cow, cat, dog, rat or mouse. In specific embodiments, the animal is a human.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. Thus, one of skill in the art understands that the term “antibody” refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. (See, e.g., Antibodies: A Laboratory Manual, Cold Spring Harbor Laboratory, 1988).

As used herein, the term “conception” refers to the union of the male sperm and the ovum of the female, also known as fertilization.

As used herein, the term “contraception” refers to the prevention of conception. A contraceptive device, thus, refers to any process, device, or method that prevents conception, development of the pre-implantation embryo, and/or implantation. Well known categories of contraceptives include, steroids, chemical barrier, physical barrier; combinations of chemical and physical barriers; abstinence and permanent surgical procedures. Contraceptives can be administered to either males or females.

As used herein, the term “DNA” is defined as deoxyribonucleic acid.

As used herein, the term “DNA segment” refers to a DNA molecule that has been isolated free of total genomic DNA of a particular species. Included within the term “DNA segment” are DNA segments and smaller fragments of such segments, and also recombinant vectors, including, for example, plasmids, cosmids, phage, viruses, and the like.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of MRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat infertility. Yet further, the present invention utilizes the expression construct or transgene as a “prophylactic construct” for contraception. Thus, the “prophylactic construct” is a contraceptive.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra.

As used herein, the term “gene” is used for simplicity to refer to nucleic acid sequences that encode a functional protein, polypeptide or peptide. This functional term includes both genomic sequences, cDNA sequences and engineered segments that express, or may be adapted to express, proteins, polypeptides, domains, peptides, fusion proteins and mutant.

As used herein, the term “fertility” refers to the quality of being productive or able to conceive. Fertility relates to both male and female animals.

As used herein, the term “infertility” refers to the inability or diminished ability to conceive or produce offspring. Infertility can be present in either male or female. In the present invention, administration of a composition to enhance infertility or decrease fertility is reversible.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic and/or prophylactic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “purified protein or peptide”, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

As used herein, the term “RNA” is defined as ribonucleic acid.

As used herein, the term “RNA interference” or “RNAi” is an RNA molecule that is used to inhibit a particular gene of interest.

As used herein, the term “under transcriptional control” or “operatively linked” is defined as the promoter is in the correct location and orientation in relation to the nucleic acid to control RNA polymerase initiation and expression of the gene.

The present invention used an in silico (electronic database) subtraction to identify a new member of the Ret Finger Protein-Like gene family, Rfpl4. Rfpl4 encodes a 287 amino acid putative E3 ubiquitin-protein ligase with a RING finger-like domain and a B30.2 motif. RT-PCR and Northern blot analyses revealed that Rfpl4 encodes a 1.7 kb mRNA detectable exclusively in the gonads of adult mice. In situ hybridization localized Rfpl4 transcripts within the ovary to oocytes of primary and later stage follicles and in the testis to elongating spermatids. The Rfpl4 gene is comprised of 3 exons and maps to mouse chromosome 7. The human ortholog was mapped to chromosome 19ql3.4. As used herein, one of skill in the field understands that “Rfpl4” denotes a mouse gene and “RFPL4” denotes a human gene. However, the scope of the present invention covers any vertebrate RFPL4 gene or protein and should not be limited to a mouse or human gene or protein. Thus, as used herein Rfpl4 and RFPL4 or any other annotation of RFPL4 is within the scope of the present invention and are interchangeable.

II. RFPL4 Protein

The protein sequence for human RFPL4 is provided in SEQ.ID.NO: 3, SEQ.ID.NO: 4 or SEQ.ID.NO: 5. In addition to the entire RFPL4 molecule, the present invention also relates to fragments of the polypeptides that may or may not retain the functions described below. Fragments, including the N-terminus of the molecule, may be generated by genetic engineering of translation stop sites within the coding region. Alternatively, treatment of the RFPL4 polypeptides with proteolytic enzymes, known as proteases, can produce a variety of N-terminal, C-terminal and internal fragments. Examples of fragments may include contiguous residues of SEQ.ID.NO: 3, SEQ.ID.NO: 4, and/or SEQ.ID.NO: 5 of 5 to 300 or more amino acids in length or any variation thereof. For example, the fragments can include, but are not limited to 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 75, 80, 85, 90, 95, 100, 200 or more amino acids in length any variation therebetween. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Variants of RFPL4

Amino acid sequence variants of the RFPL4 polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

B. Domain Switching

Yet further, the present invention has identified murine and human RFPL4 polypeptides. An interesting series of mutants can be created by substituting homologous regions of various proteins. This is known, in certain contexts, as “domain switching.”

Domain switching involves the generation of chimeric molecules using different but, in this case, related polypeptides. By comparing various RFPL4 proteins or polypeptides, one can make predictions as to the functionally significant regions of these molecules. It is possible, then, to switch related domains of these molecules in an effort to determine the criticality of these regions to RFPL4 function. These molecules may have additional value in that these “chimeras” can be distinguished from natural molecules, while possibly providing the same function.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion protein includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Proteins

In specific embodiments of the present invention, it is desirable to purify RFPL4 proteins or polypeptides or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

E. Synthetic Peptides

The present invention also describes smaller RFPL4-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

F. Antigen Compositions

The present invention also provides for the use of RFPL4 proteins or polypeptides as antigens for the immunization of animals relating to the production of antibodies. It is envisioned that RFPL4 proteins, polypeptides or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

1. Antibody Production

In certain embodiments, the present invention provides antibodies that bind with high specificity to the RFPL4 polypeptides provided herein. Thus, antibodies that bind to the polypeptide of SEQ.ID.NO: 3, SEQ.ID.NO: 4 and/or SEQ.ID.NO: 5 are provided. In addition to antibodies generated against the full length proteins, antibodies may also be generated in response to smaller constructs comprising epitopic core regions, including wild-type and mutant epitopes.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, humanized antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

A polyclonal antibody is prepared by immunizing an animal with an immunogenic RFPL4 composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, Pa.); low-dose Cyclophosphamide (CYP; 300 mg/m2) (Johnson/ Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified RFPL4 protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding pp. 71-74, 1986).

Fusion procedures usually produce viable hybrids at low frequencies, about 1×10−⁶ to 1×10−⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azasenne. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

2. Antibody Conjugates

The present invention further provides antibodies against RFPL4, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Antibody conjugates are thus preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Again, antibody-directed imaging is less preferred for use with this invention.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention ²¹¹astatine, ¹⁴carbon, ⁵¹chromium, ³⁶chlorine, ⁵⁷cobalt, ⁵⁸cobalt, 67copper, ¹⁵²Eu, ⁶⁷gallium, ³hydrogen, ¹²³iodine, ¹³¹iodine, ¹¹¹indium, ⁵⁹iron, ³²phosphorus, ¹⁸⁶rhenium, ¹⁸⁸rhenium, ⁷⁵selenium, ³⁵sulphur, and ^{99m}technicium. ¹²⁵I is often being preferred for use in certain embodiments, and ^{99m}techniciumand ¹¹¹indium are also often preferred due to their low energy and suitability for long range detection.

Radioactively labeled monoclonal antibodies of the present invention may be produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with ^{99m}technetium by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA). Also contemplated for use are fluorescent labels, including rhodamine, fluorescein isothiocyanate and renographin.

The much preferred antibody conjugates of the present invention are those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

III. RFPL4 Nucleic Acids

Important aspects of the present invention concern isolated DNA segments and recombinant vectors encoding RFPL4 proteins, polypeptides or peptides, and the creation and use of recombinant host cells through the application of DNA technology, that express a wild-type, polymorphic or mutant RFPL4, using nucleic acid sequences of SEQ.ID.NO: 1, SEQ.ID.NO: 2, or SEQ.ID.NO: 6, and biologically functional equivalents thereof.

The present invention concerns DNA segments, isolatable from mammalian cells, such as mouse, rat or human cells, that are free from total genomic DNA and that are capable of expressing a protein, polypeptide or peptide. Therefore, a DNA segment encoding RFPL4 refers to a DNA segment that contains wild-type, polymorphic or mutant RFPL4 coding sequences yet is isolated away from, or purified free from, total mammalian genomic DNA.

Similarly, a DNA segment comprising an isolated or purified RFPL4 gene refers to a DNA segment encoding RFPL4 protein, polypeptide or peptide coding sequences and, in certain aspects, regulatory sequences, isolated substantially away from other naturally-occurring genes or protein encoding sequences. As will be understood by those in the art, this functional term gene includes both genomic sequences, cDNA sequences and engineered segments that express, or may be adapted to express, proteins, polypeptides, domains, peptides, fusion proteins and mutants of RFPL4 encoded sequences.

Isolated substantially away from other coding sequences means that the gene of interest, in this case the RFPL4 gene, forms the significant part of the coding region of the DNA segment, and that the DNA segment does not contain large portions of naturally-occurring coding DNA, such as large chromosomal fragments or other functional genes or CDNA coding regions. Of course, this refers to the DNA segment as originally isolated, and does not exclude genes or coding regions later added to the segment by the hand of man.

A. Variants

In particular embodiments, the invention concerns isolated DNA segments and recombinant vectors incorporating DNA sequences that encode an RFPL4 protein, polypeptide or peptide that includes within its amino acid sequence a contiguous amino acid sequence in accordance with, or essentially as set forth in, SEQ.ID.NO: 3, SEQ.ID.NO: 4 or SEQ.ID.NO: 5, such that the sequence substantially corresponds to a portion of SEQ.ID.NO: 3, SEQ.ID.NO: 4 or SEQ.ID.NO: 5 and has relatively few amino acids that are not identical to, or a biologically functional equivalent of, the amino acids of SEQ.ID.NO: 3, SEQ.ID.NO: 4 or SEQ.ID.NO: 5.

Thus, in particular embodiments, the biological activity of an RFPL4 protein, polypeptide or peptide, or a biologically functional equivalent, for example, is transferring ubiquitin polymers from E2 ubiquitin-conjugating enzymes to recipient proteins that are then marked for proteolysis, particularly E3 ubiquitin-protein ligases.

In certain other embodiments, the invention concerns isolated DNA segments and recombinant vectors that include within their sequence a nucleic acid sequence essentially as set forth in SEQ.ID.NO: 1, SEQ.ID.NO: 2, or SEQ.ID.NO: 6. The term essentially as set forth in SEQ.ID.NO: 1, SEQ.ID.NO: 2, or SEQ.ID.NO: 6 is used in the same sense as described above and means that the nucleic acid sequence substantially corresponds to a portion of SEQ.ID.NO: 1, SEQ.ID.NO: 2, or SEQ.ID.NO: 6 and has relatively few codons that are not identical, or functionally equivalent, to the codons of SEQ.ID.NO: 1, SEQ.ID.NO: 2, or SEQ.ID.NO: 6.

Functionally equivalent codons are codons that encode the same amino acid, such as the six codons for arginine and serine, and it also refers to codons that encode biologically equivalent amino acids. Codon usage for various organisms and organelles is well known in the art, thus allowing one of skill in the art to optimize codon usage for expression in various organisms using the disclosures herein. It is contemplated that codon usage may be optimized for the desired animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria or chloroplasts, based on the preferred codon usage as would be known to those of ordinary skill in the art.

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

B. Nucleic Acid Hybridization

The nucleic acid sequences disclosed herein also have a variety of uses, such as for example, utility as probes or primers in nucleic acid hybridization embodiments.

Naturally, the present invention also encompasses DNA segments that are complementary, or essentially complementary, to the sequence set forth in SEQ.ID.NO: 1, SEQ.ID.NO: 2, and SEQ.ID.NO: 6. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment of SEQ.ID.NO: 1, SEQ.ID.NO: 2, or SEQ.ID.NO: 6 under stringent conditions such as those described herein.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl2, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl2, at temperatures ranging from approximately 40° C. to about 72° C.

Accordingly, the nucleotide sequences of the disclosure may be used for their ability to selectively form duplex molecules with complementary stretches of genes or RNAs or to provide primers for amplification of DNA or RNA from tissues. Depending on the application envisioned, it is preferred to employ varying conditions of hybridization to achieve varying degrees of selectivity of probe towards target sequence.

The nucleic acid segments of the present invention, regardless of the length of the coding sequence itself, may be combined with other DNA sequences, such as promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

The use of a hybridization probe of between 17 and 100 nucleotides in length, or in some aspect of the invention even up to 1-2 Kb or more in length, allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

In general, it is envisioned that the hybridization probes described herein will be useful both as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the “G+C” content, type of target nucleic acid, source of nucleic acid, size of hybridization probe, etc.). Following washing of the hybridized surface to remove non-specifically bound probe molecules, hybridization is detected, or even quantified, by means of the label.

C. Nucleic Acid Amplification

Nucleic acid used as a template for amplification is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to RFPL4 genes are contacted with the isolated nucleic acid under conditions that permit selective hybridization. The term “primer,” as defined herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals.

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, each incorporated herein by reference in entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641, incorporated herein by reference. Polymerase chain reaction methodologies are well known in the art.

Another method for amplification is the ligase chain reaction (“LCR”), disclosed in EPA No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, incorporated herein by reference, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be usefull in the amplification of nucleic acids in the present invention.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Gingeras et al., PCT Application WO 88/10315, incorporated herein by reference). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EP 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990).

Methods based on ligation of two (or more) oligonucleotides in the presence of nucleic acid having the sequence of the resulting “di-oligonucleotide”, thereby amplifying the di-oligonucleotide, may also be used in the amplification step of the present invention.

D. Nucleic Acid Detection

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means visible to the human eye or spectrophotometrically, to identify specific hybridization with complementary nucleic acid-containing samples.

In embodiments wherein nucleic acids are amplified, it is desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by Southern blot and hybridization analysis with a labeled probe. The techniques involved in Southern blot analysis are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

IV. Engineering Expression Constructs

In certain embodiments, the present invention involves the manipulation of genetic material to produce expression constructs that encode an RFPL4 nucleic acid sequence or gene. Such methods involve the generation of expression constructs containing, for example, a heterologous DNA encoding a gene of interest and a means for its expression, replicating the vector in an appropriate helper cell, obtaining viral particles produced therefrom, and infecting cells with the recombinant virus particles.

The gene will be a normal RFPL4 gene discussed herein above. In the context of gene therapy, the gene will be a heterologous DNA, meant to include DNA derived from a source other than the viral genome which provides the backbone of the vector. The gene may be derived from a prokaryotic or eukaryotic source such as a bacterium, a virus, a yeast, a parasite, a plant, or even an animal. The heterologous DNA also may be derived from more than one source, i.e., a multigene construct or a fusion protein. The heterologous DNA also may include a regulatory sequence which may be derived from one source and the gene from a different source.

A. Selectable Markers

In certain embodiments of the invention, the therapeutic expression and/or prophylactic constructs of the present invention contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) are employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, βgal or chloramphenicol acetyltransferase (CAT).

B. Control Regions

1. Promoters

The particular promoter employed to control the expression of a polynucleotide sequence of interest is not believed to be important, so long as it is capable of directing the expression of the polynucleotide in the targeted cell. Thus, where a human cell is targeted, it is preferable to position the polynucleotide sequence coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene product are toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Similarly tissue specific promoters are used to effect transcription in specific tissues or cells so as to reduce potential toxicity or undesirable effects to non-targeted tissues. For example, promoters such as an oocyte-specific promoter: Zp3 promoter (Lira et al., 1990), a spermatocyte-specific promoter: PGK2 promoter (Zhang et al., 1999); and a spermatid-specific promoter: Protamine promoter (Peschon et al., 1987).

In certain indications, it is desirable to activate transcription at specific times after administration of the gene therapy vector. This is done with such promoters as those that are hormone or cytokine regulatable. Cytokine and inflammatory protein responsive promoters that can be used include K and T Kininogen (Kageyama et al., 1987), c-fos, TNF-alpha, C-reactive protein (Arcone et al., 1988), haptoglobin (Oliviero et aL, 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that any of the above promoters alone or in combination with another can be useful according to the present invention depending on the action desired. In addition, this list of promoters should not be construed to be exhaustive or limiting, those of skill in the art will know of other promoters that are used in conjunction with the promoters and methods disclosed herein.

2. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Any promoter/enhancer combination (as per the Eukaryotic Promoter Data Base EPDB) can be used to drive expression of the gene. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence is employed such as human or bovine growth hormone and SV40 polyadenylation signals. Also contemplated as an element of the expression cassette is a terminator. These elements can serve to enhance message levels and to minimize read through from the cassette into other sequences.

4. Integration Sequences

In instances wherein it is beneficial that the expression vector replicate in a cell, the vector may integrate into the genome of the cell by way of integration sequences, i.e., retrovirus long terminal repeat sequences (LTRs), the adeno-associated virus ITR sequences, which are present in the vector, or alternatively, the vector may itself comprise an origin of DNA replication and other sequence which facilitate replication of the vector in the cell while the vector maintains an episomal form. For example, the expression vector may optionally comprise an Epstein-Barr virus (EBV) origin of DNA replication and sequences which encode the EBV EBNA-1 protein in order that episomal replication of the vector is facilitated in a cell into which the vector is introduced. For example, DNA constructs having the EBV origin and the nuclear antigen EBNA-1 coding are capable of replication to high copy number in mammalian cells and are commercially available from, for example, Invitrogen (San Diego, Calif.).

It is important to note that in the present invention it is not necessary for the expression vector to be integrated into the genome of the cell for proper protein expression. Rather, the expression vector may also be present in a desired cell in the form of an episomal molecule. For example, there are certain cell types in which it is not necessary that the expression vector replicate in order to express the desired protein. These cells are those which do not normally replicate and yet are fully capable of gene expression. An expression vector is introduced into non-dividing cells and express the protein encoded thereby in the absence of replication of the expression vector.

V. Methods of Gene Transfer

In order to mediate the effect of the transgene expression in a cell, it will be necessary to transfer the expression constructs of the present invention into a cell. Such transfer may employ viral or non-viral methods of gene transfer. This section provides a discussion of methods and compositions of gene transfer.

A. Non-viral Transfer

Several non-viral methods for the transfer of expression constructs into cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

In a specific embodiment of the present invention, the expression construct is complexed to a cationic polymer. Cationic polymers, which are water-soluble complexes, are well known in the art and have been utilized as a delivery system for DNA plasmids. This strategy employs the use of a soluble system, which will convey the DNA into the cells via a receptor-mediated endocytosis (Wu & Wu 1988). One skilled in the art realizes that the complexing nucleic acids with a cationic polymer will help neutralize the negative charge of the nucleic acid allowing increased endocytic uptake.

In a particular embodiment of the invention, the expression construct is entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection. Also included are various commercial approaches involving “lipofection” technology.

In certain embodiments of the invention, the liposome is complexed with a hemagglutinating virus (HVJ). This has been shown to facilitate fusion with the cell membrane and promote cell entry of liposome-encapsulated DNA (Kaneda et al., 1989). In other embodiments, the liposome is complexed or employed in conjunction with nuclear nonhistone chromosomal proteins (HMG-1) (Kato et al., 1991). In yet further embodiments, the liposome is complexed or employed in conjunction with both HVJ and HMG-1. In that such expression constructs have been successfully employed in transfer and expression of nucleic acid in vitro and in vivo, then they are applicable for the present invention.

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also is specifically delivered into a cell type such as prostate, epithelial or tumor cells, by any number of receptor-ligand systems with or without liposomes. For example, the human prostate-specific antigen (Watt et al., 1986) is used as the receptor for mediated delivery of a nucleic acid in prostate tissue.

In another embodiment of the invention, the expression construct may simply consist of naked recombinant DNA or plasmids. Transfer of the construct is performed by any of the methods mentioned above which physically or chemically permeabilize the cell membrane. This is applicable particularly for transfer in vitro, however, it is applied for in vivo use as well. Dubensky et al., (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM also is transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

B. Viral Vector-Mediated Transfer

In certain embodiments, transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

1. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

2. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

3. Adeno-associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1995 ; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

4. Other Viral Vectors

Other viral vectors are employed as expression constructs in the present invention. Vectors derived from viruses such as vaccinia virus (Ridgeway, 1988; Baichwal and Sugden, 1986; Coupar et al., 1988) canary pox virus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

VI. Mutagenesis, Peptidomimetics and Rational Drug Design

It will also be understood that this invention is not limited to the particular nucleic acid and amino acid sequences of the present invention. Recombinant vectors and isolated DNA segments may therefore include these coding regions themselves, coding regions bearing selected alterations or modifications in the basic coding region, or they may encode larger polypeptides that nevertheless include such coding regions or may encode biologically functional equivalent proteins, polypeptides or peptides that have variant amino acids sequences.

The DNA segments of the present invention encompass biologically functional equivalent RFPL4 proteins, polypeptides, and peptides. Such sequences may arise as a consequence of codon redundancy and functional equivalency that are known to occur naturally within nucleic acid sequences and the proteinaceous compositions thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes may be introduced, for example, through the application of site-directed mutagenesis techniques as discussed herein below, e.g., to introduce improvements to the antigenicity of the proteinaceous composition or to test mutants in order to examine RFPL4 activity at the molecular level.

Site-specific mutagenesis is a technique useful in the preparation of individual peptides, or biologically functional equivalent proteins, polypeptides or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, site-directed mutagenesis is performed by first obtaining a single-stranded vector, or melting of two strands of a double stranded vector, which includes within its sequence a DNA sequence encoding the desired proteinaceous molecule. An oligonucleotide primer bearing the desired mutated sequence is synthetically prepared. This primer is then annealed with the single-stranded DNA preparation, and subjected to DNA polymerizing enzymes such as E. coli polymerase I Klenow fragment or Taq polymerase, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants or PCR™ methods can be used to obtain sequence variants

As modifications and changes may be made in the structure of the RFPL4 genes, nucleic acids (e.g., nucleic acid segments) and proteinaceous molecules of the present invention, and still obtain molecules having like or otherwise desirable characteristics, such biologically functional equivalents are also encompassed within the present invention.

Equally, the same considerations may be employed to create a protein, polypeptide or peptide with countervailing, e.g., antagonistic properties. This is relevant to the present invention in which RFPL4 mutants or analogues may be generated. For example, an RFPL4 mutant may be generated and tested for RFPL4 activity to identify those residues important for RFPL4 activity. RFPL4 mutants may also be synthesized to reflect an RFPL4 mutant that occurs in the human population and that is linked to infertility. Such mutant proteinaceous molecules are particularly contemplated for use in generating mutant-specific antibodies and such mutant DNA segments may be used as mutant-specific probes and primers.

In terms of functional equivalents, it is well understood by the skilled artisan that, inherent in the definition of a biologically functional equivalent protein, polypeptide, peptide, gene or nucleic acid, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted.

In particular, where shorter length peptides are concerned, it is contemplated that fewer amino acids changes should be made within the given peptide. Longer domains may have an intermediate number of changes. The full length protein will have the most tolerance for a larger number of changes. Of course, a plurality of distinct proteins/polypeptide/peptides with different substitutions may easily be made and used in accordance with the invention.

It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity.

In addition to the RFPL4 peptidyl compounds described herein, it is contemplated that other sterically similar compounds may be formulated to mimic the key portions of the peptide structure. Such compounds, which may be termed peptidomimetics, may be used in the same manner as the peptides of the invention and hence are also functional equivalents.

Certain mimetics that mimic elements of proteinaceous molecule's secondary structure are described in Johnson et al. (1993). The underlying rationale behind the use of peptide mimetics is that the peptide backbone of proteinaceous molecules exists chiefly to orientate amino acid side chains in such a way as to facilitate molecular interactions, such as those of antibody and antigen. A peptide mimetic is thus designed to permit molecular interactions similar to the natural molecule.

Some successful applications of the peptide mimetic concept have focused on mimetics of β-turns within proteinaceous molecules, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

The generation of further structural equivalents or mimetics may be achieved by the techniques of modeling and chemical design known to those of skill in the art. The art of receptor modeling is now well known, and by such methods a chemical that binds RFPL4 can be designed and then synthesized. It will be understood that all such sterically designed constructs fall within the scope of the present invention.

In one aspect, a compound may be designed by rational drug design to function as a modulator of E3 ubiquitin-protein ligase. The goal of rational drug design is to produce structural analogs of biologically active compounds. By creating such analogs, it is possible to fashion drugs, which are more active or stable than the natural molecules, which have different susceptibility to alteration or which may affect the function of various other molecules. In one approach, one would generate a three-dimensional structure for the RFPL4 protein of the invention or a fragment thereof. This could be accomplished by X-ray crystallography, computer modeling or by a combination of both approaches. An alternative approach, involves the random replacement of functional groups throughout the RFPL4 protein, polypeptides or peptides, and the resulting affect on function determined.

It also is possible to isolate an RFPL4 protein, polypeptide or peptide specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically—produced peptides. Selected peptides would then serve as the pharmacore. Anti-idiotypes may be generated using the methods described herein for producing antibodies, using an antibody as the antigen.

Thus, one may design drugs which have enhanced and improved biological activity, for example, E3 ubiquitin-protein ligase activity, contraception, enhanced fertility, relative to a starting RFPL4 proteinaceous sequences. By virtue of the ability to recombinatly produce sufficient amounts of the RFPL4 proteins, polypeptides or peptides, crystallographic studies may be preformed to determine the most likely sites for mutagenesis and chemical mimicry. In addition, knowledge of the chemical characteristics of these compounds permits computer employed predictions of structure-function relationships. Computer models of various polypeptide and peptide structures are also available in the literature or computer databases. In a non-limiting example, the Entrez database may be used by one of ordinary skill in the art to identify target sequences and regions for mutagenesis.

VII. Methods for Screening Modulators

The present invention also contemplates the use of RFPL4 and active fragments, and nucleic acids coding therefor, in the screening of compounds for activity in either stimulating RFPL4 activity, overcoming the lack of RFPL4 or blocking or inhibiting the effect of an RFPL4 molecule. These assays may make use of a variety of different formats and may depend on the kind of “activity” for which the screen is being conducted.

A. In vitro Assays

In one embodiment, the invention is to be applied for the screening of compounds that bind to the RFPL4 polypeptide or fragment thereof. The polypeptide or fragment may be either free in solution, fixed to a support, expressed in or on the surface of a cell. Either the polypeptide or the compound may be labeled, thereby permitting determining of binding.

In another embodiment, the assay may measure the inhibition of binding of RFPL4 to a natural or artificial substrate or binding partner. Competitive binding assays can be performed in which one of the agents (RFPL4, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with RFPL4 and washed. Bound polypeptide is detected by various methods.

Purified RFPL4 can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the RFPL4 active region to a solid phase.

Various cell lines containing wild-type or natural or engineered mutations in RFPL4 gene can be used to study various functional attributes of RFPL4 and how a candidate compound affects these attributes. Methods for engineering mutations are described elsewhere in this document, as are naturally-occurring mutations in RFPL4 that lead to, contribute to and/or otherwise cause infertility. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of RFPL4, or related pathways, may be explored.

In a specific embodiment, yeast two-hybrid analysis is performed by standard means in the art with the polypeptides of the present invention, i.e., RFPL4. Two hybrid screen is used to elucidate or characterize the function of a protein by identifying other proteins with which it interacts. The protein of unknown function, herein referred to as the “bait” is produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the bait protein and the potential target protein in a particular cell, the GAL4 components remain separate and unable to promote reporter gene transcription on their own. One skilled in the art is aware that different reporter genes can be utilized, including β-galactosidase, HIS3, ADE2, or URA3. Furthermore, multiple reporter sequences, each under the control of a different inducible promoter, can be utilized within the same cell to indicate interaction of the GAL4 components (and thus a specific bait and target protein). A skilled artisan is aware that use of multiple reporter sequences decreases the chances of obtaining false positive candidates. Also, alternative DNA-binding domain/activation domain components may be used, such as LexA. One skilled in the art is aware that any activation domain may be paired with any DNA binding domain so long as they are able to generate transactivation of a reporter gene. Furthermore, a skilled artisan is aware that either of the two components may be of prokaryotic origin, as long as the other component is present and they jointly allow transactivation of the reporter gene, as with the LexA system.

Two hybrid experimental reagents and design are well known to those skilled in the art (see The Yeast Two-Hybrid System by P. L. Bartel and S. Fields (eds.) (Oxford University Press, 1997), including the most updated improvements of the system (Fashena et al, 2000). A skilled artisan is aware of commercially available vectors, such as the Matchmaker™ Systems from Clontech (Palo Alto, Calif.) or the HybriZAP® 2.1 Two Hybrid System (Stratagene; La Jolla, Calif.), or vectors available through the research community (Yang et al., 1995; James et al., 1996). In alternative embodiments, organisms other than yeast are used for two hybrid analysis, such as mammals (Mammalian Two Hybrid Assay Kit from Stratagene (La Jolla, Calif.)) or E. coli (Hu et al., 2000).

In an alternative embodiment, a two hybrid system is utilized wherein protein-protein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System from Stratagene (La Jolla, Calif.), in which a target protein becomes anchored to a cell membrane of a yeast which contains a temperature sensitive mutation in the cdc25 gene, the yeast homologue for hSos (a guanyl nucleotide exchange factor). Upon binding of a bait protein to the target, hSos is localized to the membrane, which allows activation of RAS by promoting GDP/GTP exchange. RAS then activates a signaling cascade which allows growth at 37° C. of a mutant yeast cdc25H. Vectors (such as pMyr and psos) and other experimental details are available for this system to a skilled artisan through Stratagene (La Jolla, Calif.). (See also, for example, U.S. Pat. No. 5,776,689, herein incorporated by reference).

Thus, in accordance with an embodiment of the present invention, there is a method of screening for a peptide which interacts with RFPL4 comprising introducing into a cell a first nucleic acid comprising a DNA segment encoding a test peptide, wherein the test peptide is fused to a DNA binding domain, and a second nucleic acid comprising a DNA segment encoding at least part of RFPL4, respectively, wherein the at least part of RFPL4 respectively, is fused to a DNA activation domain. Subsequently, there is an assay for interaction between the test peptide and the RFPL4 polypeptide or fragment thereof by assaying for interaction between the DNA binding domain and the DNA activation domain. For example, the assay for interaction between the DNA binding and activation domains may be activation of expression of β-galactosidase.

An alternative method is screening of lambda.gt11, lambda.LZAP (Stratagene) or equivalent cDNA expression libraries with recombinant RFPL4. Recombinant RFPL4 or fragments thereof are fused to small peptide tags such as FLAG, HSV or GST. The peptide tags can possess convenient phosphorylation sites for a kinase such as heart muscle creatine kinase or they can be biotinylated. Recombinant RFPL4 can be phosphorylated with ³²[P] or used unlabeled and detected with streptavidin or antibodies against the tags. lambda.gt11cDNA expression libraries are made from cells of interest and are incubated with the recombinant RFPL4, washed and cDNA clones which interact with RFPL4 isolated. Such methods are routinely used by skilled artisans. See, e.g., Sambrook (supra).

Another method is the screening of a mammalian expression library in which the cDNAs are cloned into a vector between a mammalian promoter and polyadenylation site and transiently transfected in cells. Forty-eight hours later the binding protein is detected by incubation of fixed and washed cells with a labeled RFPL4. In this manner, pools of cDNAs containing the cDNA encoding the binding protein of interest can be selected and the cDNA of interest can be isolated by further subdivision of each pool followed by cycles of transient transfection, binding and autoradiography. Alternatively, the cDNA of interest can be isolated by transfecting the entire cDNA library into mammalian cells and panning the cells on a dish containing the RFPL4 bound to the plate. Cells which attach after washing are lysed and the plasmid DNA isolated, amplified in bacteria, and the cycle of transfection and panning repeated until a single cDNA clone is obtained. See Seed et al., 1987 and Aruffo et al., 1987 which are herein incorporated by reference. If the binding protein is secreted, its cDNA can be obtained by a similar pooling strategy once a binding or neutralizing assay has been established for assaying supernatants from transiently transfected cells. General methods for screening supernatants are disclosed in Wong et al., (1985).

Another alternative method is isolation of proteins interacting with the RFPL4 directly from cells. Fusion proteins of RFPL4 with GST or small peptide tags are made and immobilized on beads. Biosynthetically labeled or unlabeled protein extracts from the cells of interest are prepared, incubated with the beads and washed with buffer. Proteins interacting with the RFPL4 are eluted specifically from the beads and analyzed by SDS-PAGE. Binding partner primary amino acid sequence data are obtained by microsequencing. Optionally, the cells can be treated with agents that induce a functional response such as tyro sine phosphorylation of cellular proteins. An example of such an agent would be a growth factor or cytokine such as interleukin-2.

Another alternative method is immunoaffinity purification. Recombinant RFPL4 is incubated with labeled or unlabeled cell extracts and immunoprecipitated with anti-RFPL4 antibodies. The immunoprecipitate is recovered with protein A-Sepharose and analyzed by SDS-PAGE. Unlabeled proteins are labeled by biotinylation and detected on SDS gels with streptavidin. Binding partner proteins are analyzed by microsequencing. Further, standard biochemical purification steps known to those skilled in the art may be used prior to microsequencing.

Yet another alternative method is screening of peptide libraries for binding partners. Recombinant tagged or labeled RFPL4 is used to select peptides from a peptide or phosphopeptide library which interact with the RFPL4. Sequencing of the peptides leads to identification of consensus peptide sequences which might be found in interacting proteins.

B. In Vivo Assays

The present invention also encompasses the use of various animal models. Thus, any identity seen between human and other animal RFPL4 provides an excellent opportunity to examine the function of RFPL4 in a whole animal system where it is normally expressed. By developing or isolating mutant cells lines that fail to express normal RFPL4, one can generate models in mice that enable one to study the mechanism of RFPL4 and its role in gametogenesis.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, increased fertility, decreased fertility or contraception.

In one embodiment of the invention, transgenic animals are produced which contain a functional transgene encoding a functional RFPL4 polypeptide or variants thereof. Transgenic animals expressing RFPL4 transgenes, recombinant cell lines derived from such animals and transgenic embryos may be useful in methods for screening for and identifying agents that induce or repress function of RFPL4. Transgenic animals of the present invention also can be used as models for studying disease states.

In one embodiment of the invention, an RFPL4 transgene is introduced into a non-human host to produce a transgenic animal expressing a human or murine RFPL4 gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al., 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous RFPL4 by homologous recombination between the transgene and the endogenous gene; or the endogenous gene may be eliminated by deletion as in the preparation of “knock-out” animals. Typically, an RFPL4 gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles,- amphibians, birds, mammals, and fish. Within a particularly preferred embodiment, transgenic mice are generated which overexpress RFPL4 or express a mutant form of the polypeptide. Alternatively, the absence of an RFPL4 in “knock-out” mice permits the study of the effects that loss of RFPL4 protein has on a cell in vivo.

As noted above, transgenic animals and cell lines derived from such animals may find use in certain testing experiments. In this regard, transgenic animals and cell lines capable of expressing wild-type or mutant RFPL4 may be exposed to test substances. These test substances can be screened for the ability to enhance wild-type RFPL4 expression and or function or impair the expression or function of mutant RFPL4.

VIII. Modulators of RFPL4

In certain embodiments, modulators of RFPL4 are administered to an animal to either enhance or suppress the activity and/or expression of RFPL4. It is envisioned that RFPL4 plays a role in protein degradation pathways important for gametogenesis or early embryonic development. In specific embodiments, RFPL4 plays a role in meiosis, for example, it may disrupt meiosis or it may enhance or drive meiosis.

The modulators of the present invention include, but are not limited to polynucleotides, polypeptides, antibodies, small molecules or other compositions that are capable of modulating either the activity and/or the expression of RFPL4.

A. Transcription Factors and Nuclear Binding Sites

Transcription factors are regulatory proteins that binds to a specific DNA sequence (e.g., promoters and enhancers) and regulate transcription of an encoding DNA region. Typically, a transcription factor comprises a binding domain that binds to DNA (a DNA binding domain) and a regulatory domain that controls transcription. Where a regulatory domain activates transcription, that regulatory domain is designated an activation domain. Where that regulatory domain inhibits transcription, that regulatory domain is designated a repression domain.

Activation domains, and more recently repression domains, have been demonstrated to function as independent, modular components of transcription factors. Activation domains are not typified by a single consensus sequence but instead fall into several discrete classes: for example, acidic domains in GAL4 (Ma, et al. 1987), GCN4 (Hope, et al., 1987), VP16 (Sadowski, et al. 1988), and GATA-1 (Martin, et al. 1990); glutamine-rich stretches in Sp1 (Courey, et al. 1988) and Oct-2/ OTF2 (Muller-Immergluck, et al. 1990; Gerster, et al. 1990); proline-rich sequences in CTF/NF-1 (Mermod, et al. 1989); and serine/threonine-rich regions in Pit-1/GH-F-1 (Theill, et al. 1989) all function to activate transcription. The activation domains of fos and jun are rich in both acidic and proline residues (Abate, et al. 1991; Bohmann, et al. 1989); for other activators, like the CCAAT/enhancer-binding protein C/EBP (Friedman, et al. 1990), no evident sequence motif has emerged.

B. Antisense and Ribozymes

An antisense molecule that binds to a translational or transcriptional start site, or splice junctions, are ideal modulators. Antisense, ribozyme, and double-stranded RNA molecules target a particular sequence to achieve a reduction or elimination of a particular polypeptide, such as RFPL4 or another protein that plays a role in modulating RFPL4. Thus, it is contemplated that antisense, ribozyme, and double-stranded RNA, and RNA interference molecules are constructed and used to modulate RFPL4 expression.

1. Antisense Molecules

Antisense methodology takes advantage of the fact that nucleic acids tend to pair with complementary sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Antisense constructs are designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction are used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

It is advantageous to combine portions of genomic DNA with cDNA or synthetic sequences to generate specific constructs. For example, where an intron is desired in the ultimate construct, a genomic clone will need to be used. The CDNA or a synthesized polynucleotide may provide more convenient restriction sites for the remaining portion of the construct and, therefore, would be used for the rest of the sequence.

2. Ribozymes

Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression is particularly suited to therapeutic applications (Scanlon et al., 1991; Sarver et al., 1990; Sioud et al., 1992). Most of this work involved the modification of a target MRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in RFPL4 targeted ribozymes is simply a matter of preparing and testing a given sequence, and is a routinely practiced screening method known to those of skill in the art.

3. RNA Interference

It is also contemplated in the present invention that double-stranded RNA is used as an interference molecule, e.g., RNA interference (RNAi). RNA interference is used to “knock down” or inhibit a particular gene of interest by simply injecting, bathing or feeding to the organism of interest the double-stranded RNA molecule. This technique selectively reduces the levels of the sense RNA encoded by the particular gene (Giet, 2001; Hammond, 2001; Stein P, et al., 2002; Svoboda P, et al., 2001; Svoboda P, et al., 2000).

Thus, in certain embodiments, double-stranded RFPL4 RNA is synthesized or produced using standard molecular techniques described herein. In further embodiments, double-stranded RNA molecules of other compositions that may inhibit RFPL4 are also considered and used herein.

IX. Diagnosing Infertility

As discussed above, the present inventors have determined that alterations in the RFPL4 gene are associated with infertility. Therefore, RFPL4 genes may be employed as a diagnostic or prognostic indicator of infertility in general. More specifically, point mutations, deletions, insertions or regulatory perturbations will be identified. The present invention contemplates further the diagnosis of infertility detecting changes in the levels of RFPL4 expression.

A. Genetic Diagnosis

One embodiment of the instant invention comprises a method for detecting variation in the expression of RFPL4. This may comprise determining the level of RFPL4 expressed, or determining specific alterations in the expressed product.

The biological sample can be tissue or fluid. Various embodiments include cells from the testes and ovaries. Other embodiments include fluid samples such as vaginal fluid or seminal fluid.

Nucleic acids used are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA (cDNA). In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have been diagnosed with infertility.

It is contemplated that other mutations in the RFPL4 gene may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO, e.g., U.S. Pat. No. 5,639,611), dot blot analysis, denaturing gradient gel electrophoresis (e.g., U.S. Pat. No. 5,190,856 incorporated herein by reference), RFLP (e.g., U.S. Pat. No. 5,324,631 incorporated herein by reference) and PCR™-SSCP. Methods for detecting and quantitating gene sequences, such as mutated genes and oncogenes, in for example biological fluids are described in U.S. Pat. No. 5,496,699, incorporated herein by reference.

Yet further, it is contemplated that chip-based DNA technologies such as those described by Hacia et al. (1996) and Shoemaker et al. (1996) can be used for diagnosis of infertility. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al., (1994); Fodor et al., (1991).

B. Immunodiagnosis

Antibodies can be used in characterizing the RFPL4 content through techniques such as ELISAs and Western blot analysis. This may provide a prenatal screen or in counseling for those individuals seeking to have children.

The steps of various other useful immunodetection methods have been described in the scientific literature, such as, e.g., Nakamura et al., (1987). Immunoassays, in their most simple and direct sense, are binding assays. Certain preferred immunoassays are the various types of radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

X. Methods for Treating

The present invention contemplates the use of a modulator of RFPL4 to either enhance contraception or fertility of an animal. Animals that are treated include, but are not limited to mammals or avian, for example, mice, rats, or monkeys are used as experimental animal models. In specific embodiments, the present invention is used to treat humans. It is also envisioned that companion animals can be treated for infertility or the prophylactic compositions can be used as a contraceptive. Companion animals include, but are not limited to dogs, cats, horses, or birds.

The present invention involves the method of administering a composition to animal in an amount to result in contraception or fertility. Thus, contraception involves the administration of a compound in an effective amount such that the amount decreases conception. In the present invention, any modulation or decrease in conception is considered contraception. Yet further, an amount of a compound that results in an increase in fertility is considered the effective amount.

In certain embodiments of the present invention, an effective amount of a modulator of RFPL4 is administered to an animal to enhance contraception by decreasing protein degradation. It is envisioned that inhibition of protein degradation of specific proteins results in infertility. It has been shown that specific proteins, such as, CPEB, MOS, and cyclin B1 require degradation in order for the oocyte to mature. In addition, disappearance of ZAR1 occurs at the oocyte-to-embryo transition and, thus is required at this transition (Wu et aL., Nature Genetics 2003, in press). Thus, RFPL4 may play a role in degradation of translational repressors and other factors operating within defined periods of oogenesis and/or early embryonic development.

In further specific embodiments of the present invention, an effective amount of a modulator of RFPL4 is administered to an animal to enhance contraception. It is envisioned that RFPL4 can play a role in degradation of a specific factor that allows progression thru meiosis, thus an increase in RFPL4 activity can result in infertility by disrupting meiosis. Examples of the specific factors include, but are not limited to cdc25 phosphatase.

In additional embodiments, an effective amount of a modulator of RFPL4 is administered to an animal to enhance fertility. It is envisioned that either an increase or decrease in RFPL4 can result in enhancement in fertility. Fertility is the opposite of infertility or contraception. Thus, if RFPL4 degrades a specific factor that allows progression thru meiosis, then inhibition of RFPL4 will result in an increase in fertility. Likewise, if RFPL4 is responsible for degradation of translational repressors or other factors, then an increase in RFPL4 will result in an increase in fertility.

A. Genetic Based Therapies

Specifically, the present inventors intend to provide, to a cell, an expression construct capable of enhancing or decreasing RFPL4 to that cell. Because the sequence homology between the human, and other RFPL4, any of these nucleic acids could be used in human therapy, as could any of the gene sequence variants discussed above which would encode the same, or a biologically equivalent polypeptide. The lengthy discussion of expression vectors and the genetic elements employed therein is incorporated into this section by reference. Particularly preferred expression vectors are viral vectors such as adenovirus, adeno-associated virus, herpes virus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

B. Protein Therapy

Another therapy approach is the provision, to a subject, of RFPL4 polypeptide, active fragments, synthetic peptides, mimetics or other analogs thereof. The protein may be produced by recombinant expression means. Formulations would be selected based on the route of administration and purpose including, but not limited to, liposomal formulations and classic pharmaceutical preparations.

XI. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### XII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

In Silico Subtraction

3499 EST sequences from fertilized and unfertilized egg libraries were downloaded from the NCBI database and used to perform BLAST searches of the entire EST database and the non-redundant (nr) sequence database (Rajkovic, et aL, 2001). 258 of these sequences (7.4%) were shown to match only the original egg libraries or pre-implantation embryo libraries. These included the Unigene cluster Mm.28764, later termed Rfpl4.

Rfpl4 was discovered in a search for oocyte-specific transcripts important in mammalian oogenesis. In silico subtraction was used to identify transcripts preferentially expressed in oocytes (Rajkovic et al., 2001) and found that expressed sequence tags (ESTs) from the publicly available Unigene cluster Mm.28764 were present exclusively in unfertilized egg libraries (i.e., the sequence did not appear in other EST or non-redundant public databases).

### Example 2

Sequence Analysis

Mouse Rfpl4 and human RFPL4 sequences were determined from a combination of database searches, and sequencing of the following deposited ESTs from American Type Culture Collection (Manassas, Va.): C87022, AU015407, AU016703, and AC008749. Full-length Rfpl4 cDNA was used to deduce the amino acid sequence of the RFPL4 protein. This sequence was characterized using EXPASy, PSORT, Pfam and ScanProsite programs to compare it to known protein sequences and to predict functional domains.

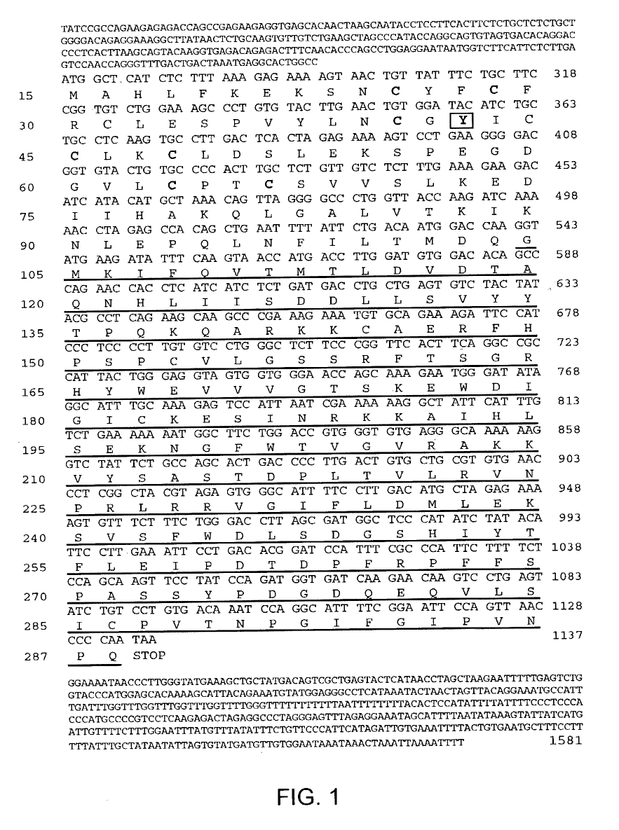

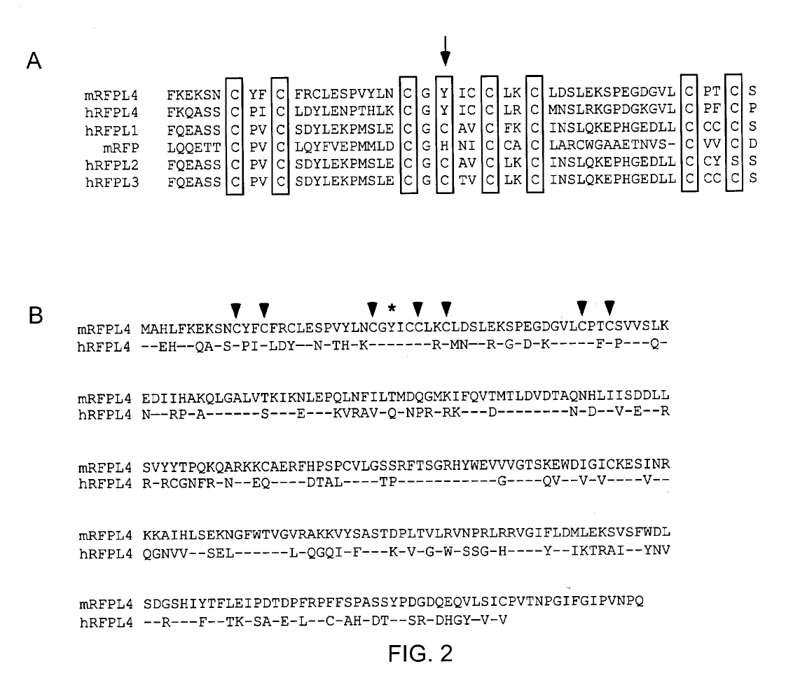

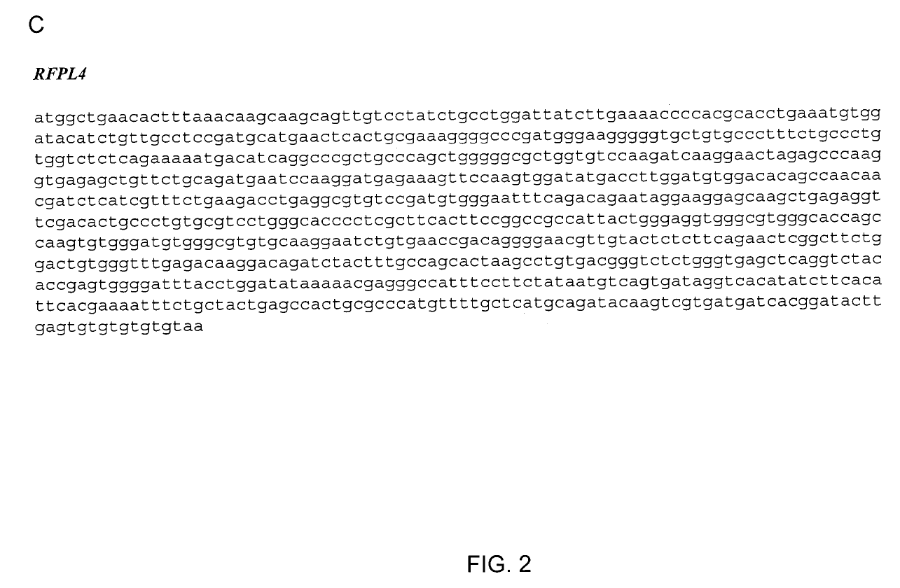

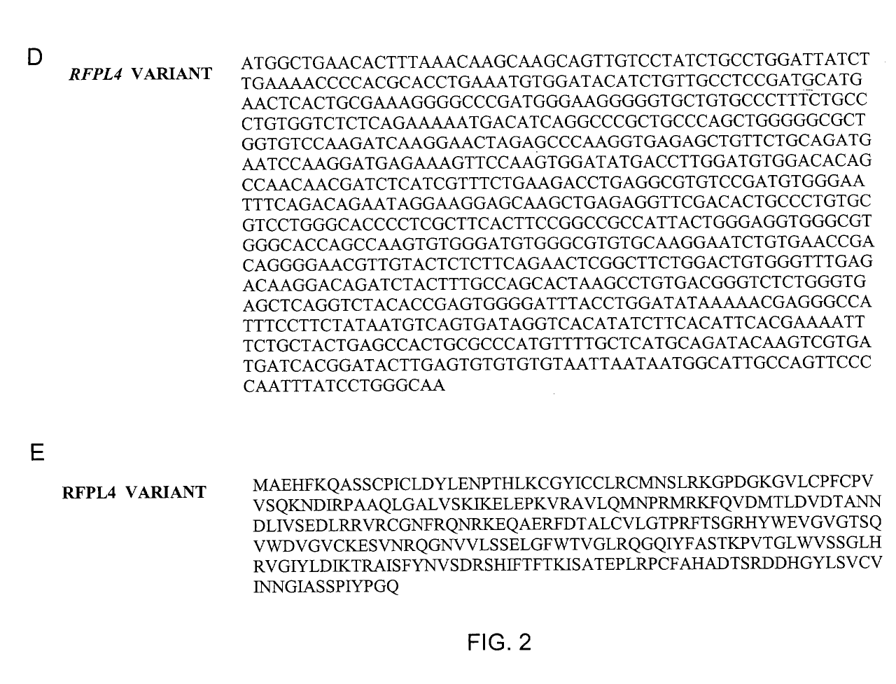

Analysis of the Rfpl4 cDNA sequence revealed a 1581 nucleotide transcript predicted to encode a 287 amino acid protein (FIG. 1). The putative RFPL4 protein had a tripartite structure consisting of a cysteine-rich RING finger-like region (C3YC4), a coiled-coiled motif, and B30.2 domains, common features of other members of the RING-B30 family, including MID1, RFP and Ro52. However, RFPL4 shared closest homology with a newly described family of human Ret Finger Protein-Like genes (RFPL1, RFPL2, and RFPL3). RFPL1, RFPL2 and RFPL3 proteins include RING finger-like motifs lacking a histidine residue (i.e., substitution of a cysteine for a histidine) common to previously described RING finger proteins (Seroussi et al., 1999). The RFPL4 protein encoded a Ret finger-like domain that also lacked this histidine residue that normally existed in the classical C3HC4 RING finger domains (FIG. 2A). A tyrosine residue was substituted in this position in both the mouse RFLP4 and the human RFPL4 ortholog (FIGS. 2A and 2B). RING finger domains were known to be important in ubiquitin-mediated proteolysis. The B30.2 domain was a conserved region of approximately 170 amino acids found in RING finger proteins, butyrophilin, stonustoxin and otherwise unrelated proteins (Henry et al., 1998; Henry et al., 1997; Seto et al., 1999).

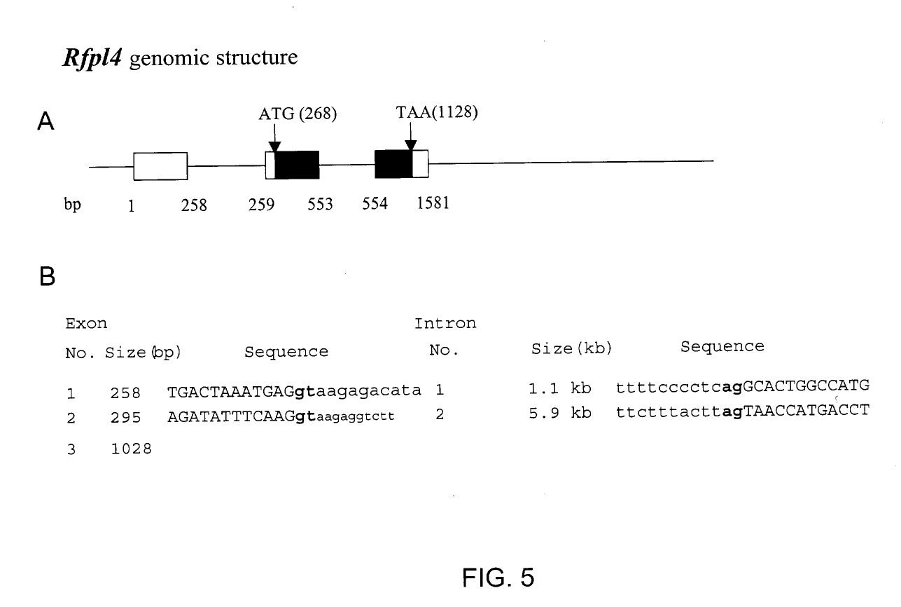

The genomic structure of the Rfpl4 gene is shown in FIG. 5. It was composed of three exons (258 bp, 295 bp and 1028 bp in length) and two intervening introns (1.1 kb and 5.9 kb in length). The ATG translation start was at position 268 in exon 2. Rfpl4 ESTs had been previously mapped to the proximal portion of mouse chromosome 7, corresponding to position 4.0 cM (Ko et al., 2000). The human RFPL4 ortholog gene mapped to 19q13.4.

### Example 3

Experimental Animals

Mice were maintained as described in the NIH Guide for the Care and Use of Laboratory Animals. Generation of mice carrying the Gdf9 null allele and Southern blot genotyping has been described (Dong, et al., 1996). Tissues were collected from adult C57BL/6/129SvEv (hybrid strain) wild-type (+/+) and Gdf9 knockout (Gdf9−/−) mice (6-16 weeks of age) for RNA isolation or in situ hybridization.

### Example 4

RT-PCR

Total RNA was isolated using an acid guanidinium thiocyanate-phenol-chloroform extraction (Leedo Medical Laboratories, Houston, Tex.). Five micrograms of RNA were reverse transcribed using the SuperScript system (Life Technologies, Rockville, Md.). Rfpl4 cDNA was then amplified using the specific primers: Rfpl4L (SEQ.ID.NO: 7 5′gggtgggagggaaaataaaa-3′) and Rfpl4R (SEQ.ID.NO: 8 5′- ggtacccatggagcacaaag-3′) as previously described (Rajkovic et al., 2001).

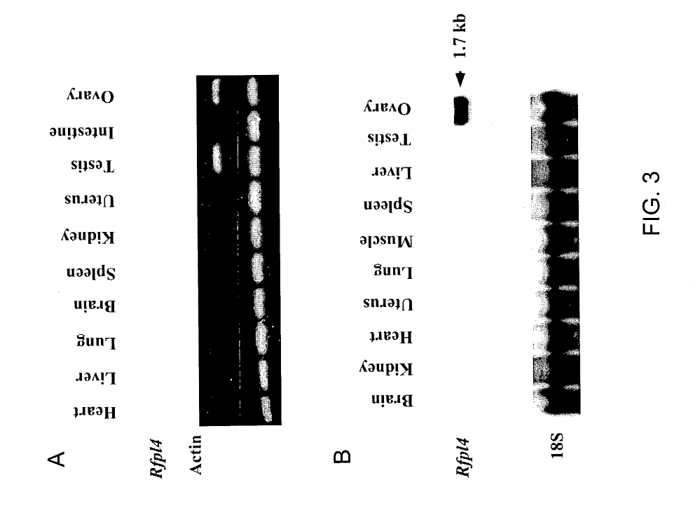

To determine the tissue distribution of Rfpl4 expression, RT-PCR and Northern hybridization analyses were performed. Oligonucleotide primers specific for Rfpl4 transcripts amplified an intervening sequence only from cDNA of adult ovaries and testes (FIG. 3A).

### Example 5

Northern Blot Analysis

Fifteen micrograms of each RNA sample was used for electrophoresis and transfer onto nylon membranes. Radioactive cDNA probes were synthesized using [α³²P]dATP and the Strip-EZ kit (Ambion Inc., Austin, Tex.). Autoradiography allowed for visualization of probe hybridization. Blots were stripped and re-probed for 18S rRNA.

Northern blot analysis revealed that the 1.7 kb Rfpl4 transcript was predominantly expressed in the ovary; the technique was not sensitive enough to detect expression in the testis (FIG. 3B).

### Example 6

In situ Hybridization

In situ hybridization was performed as previously described (Elvin et al. 1999). Briefly, [α-³⁵S]UTP-labeled antisense and sense riboprobes were transcribed from the Rfpl4 CDNA sequence (Promega Corp., Madison, Wis.). Paraffin-embedded ovaries were cut into 5 μm sections, dewaxed, fixed, hybridized, and washed as detailed (Albrecht, et al., 1997). Signal was detected by autoradiography using NTB-2 emulsion (Eastman Kodak Co., Rochester, N.Y.). Hematoxylin counter-staining allowed ready correlation of the hybridization to specific cell populations within the ovary.

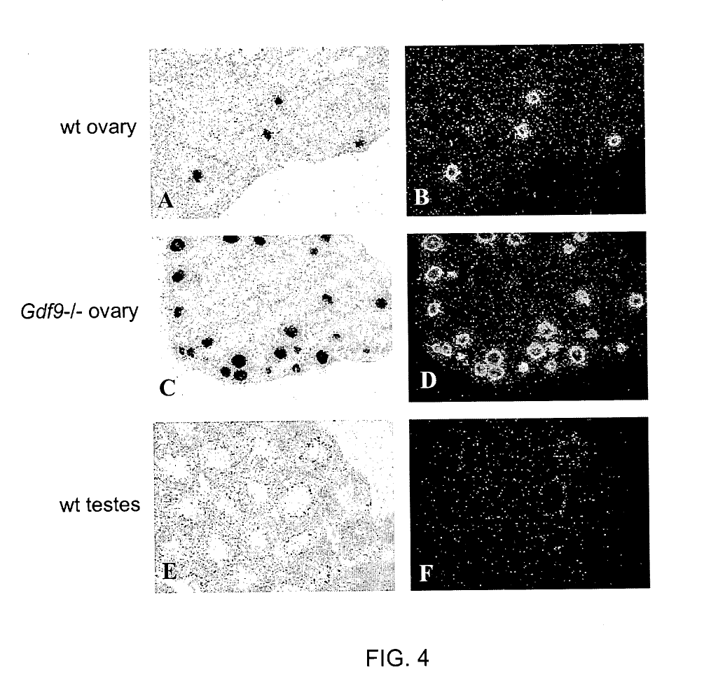

In situ hybridization showed that Rfpl4 antisense riboprobes hybridized to oocytes in one-layer (primary) follicles and oocytes of multi-layered follicles including antral follicles (FIGS. 4A and 4B). Ovaries from Gdf9 knockout mice were small and contain an abundance of oocytes in follicles arrested at the primary stage of development (Dong et al., 1996); Rfpl4 is expressed abundantly in oocytes of Gdf9 knockout ovaries (FIGS. 3B; 4C and 4D). Since Rfpl4 expression was detected in testes by semiquantitative RT-PCR, the Rfpl4 mRNA distribution was analyzed using in situ hybridization. Rfpl4 transcripts were present at later stages of spermatogenesis and were abundant in elongating spermatids (FIGS. 4E and 4F).

### Example 7

Purification of RFPL4 Protein and Specificity of the Antibody

A full-length Rfpl4 cDNA fragment was subcloned into pET-23b (Novagen, Madison, Wis.), His-tagged RFPL4 was produced in BL21 [DE3] pLysS cells, and polyclonal antibodies were raised in goats (Cocalico Biologicals Inc., Reamstown, Pa.). Immunofluorescence, Western blot, and immunohistochemical analysis were performed as described herein.

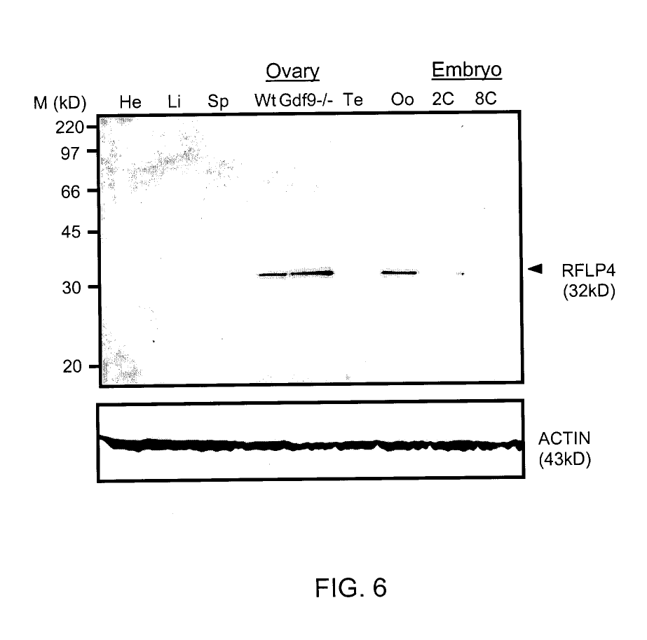

Affinity and specificity of the anti-RFPL4 antibody were tested by Western blot analysis. Total protein extracts prepared from adult mouse wild-type heart, liver, spleen, testis, ovary, and Gdf9−/− ovary (enriched in oocytes) were used. In addition, oocytes, 2-cell embryos, and 8-cell embryos were collected from the ovaries or oviducts of wild-type female mice. The RFPL4 antiserum detected RFPL4 in ovaries, oocytes, and 2-cell embryos (FIG. 6) but failed to detect a band in other samples. This finding was consistent with the Northern blot analysis showing that Rfpl4 MRNA was ovary-specific.

### Example 8

Immunohistochemical Analysis.

Immunohistochemical analysis of RFPL4 protein was performed (Elvin et al., 1999). Ovaries were fixed in 4% paraformaldehyde, embedded in paraffin, and cut to 5 μm sections. Sections were blocked in 0.1% BSA/PBS and universal blocker (BioGenex Laboratories, Inc., San Ramon, Calif.). Anti-RFPL4 antiserum or preimmune serum (control) was diluted 1:1000, and antigen detected using anti-goat IgG-biotinylated secondary antibody, streptavidin-conjugated alkaline phosphatase, and New Fuschin substrate (BioGenex Laboratories, Inc.). The slides were counterstained with hematoxylin.

### Example 9

Immunofluorescence

In brief, the expression and subcellular distribution of RFPL4 was determined in fully grown oocytes and early preimplantation embryos from (C57BL/6J×129/S6/SvEv) F1 mice. Oocytes and embryos were permeabilized, blocked, and treated with anti-RFPL4 antisera (diluted 1/500) for 1 h, washed, and incubated with rabbit anti-goat IgG (Molecular Probes, Eugene, Oreg.) for 45 min. DNA was counterstained with DAPI. Imaging was performed by deconvolution microscopy.

### Example 10

cDNA Library Construction

Ovaries were collected from adult C57BL/6/129/SvEv hybrid Gdf9 knockout (Gdf9−/−) mice (Dong et al., 1996) (6-16 weeks of age) for RNA isolation using the RNA STAT-60 reagent (Leedo Medical Laboratories, Houston, Tex.). To collect GV-stage oocytes, (C57BL/6J×SJL/J) F1 mice were sacrificed 48 hours after PMSG treatment to stimulate follicle development. Oocytes were denuded by pipetting, and mRNA was extracted using the Micro-Fast Track 2.0 kit (Invitrogen, Carlsbad, Calif.). RNA was reverse-transcribed, and cDNAs were size selected and subcloned into Sma I-linearized pGADT7-Rec cloning vector (Clontech, Palo Alto, Calif.).

### Example 11

Yeast Two-Hybrid Screen and Construction of Truncation Constructs

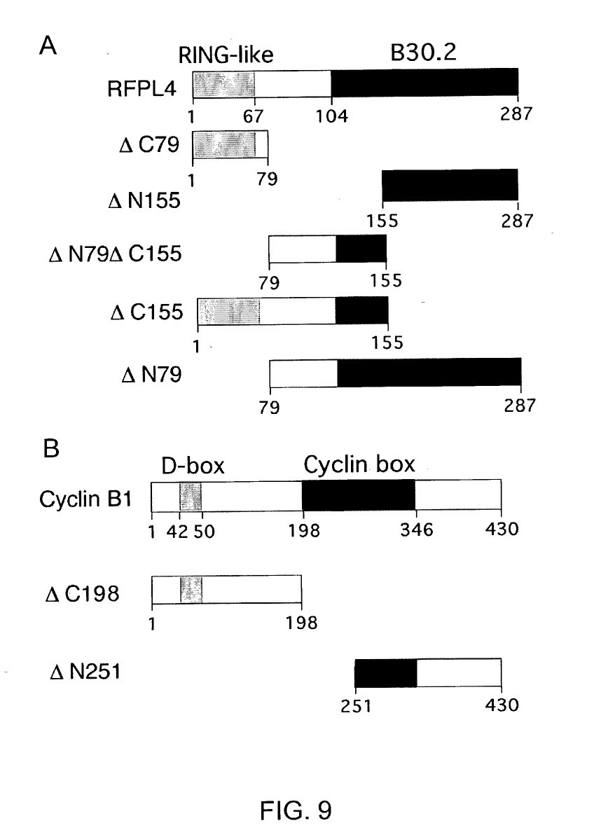

A yeast two-hybrid screen (MATCHMAKER, Clontech) was performed using pGBKT7-full-length mouse Rfpl4 cDNA (GenBank accession AY070253) as bait. After yeast mating, clones which grew on (Leu-/Trp-/Ade-/His-/X-alpha-Gal) selection plates were isolated, and candidate pGADT7-cDNAs were sequenced. For co-transformation truncation constructs, portions of Rfpl4 and cyclin B1 (Ccnb1, BC011478) were used (FIGS. 9A-9B). Expression vectors containing full-length cyclin B1 proved toxic to yeast. The full-length cDNA encoding HR6A (Ube2a, AF383148), was also subcloned into yeast expression vector. All constructs were confirmed by DNA sequencing.

### Example 12

In Vitro Transcription/Translation and Co-Immunoprecipitation

The pGBKT7 (MYC-Tagged) and pGADT7 (HA-Tagged) vectors were used as templates for in vitro transcription/translation using [³⁵S]Met and the TNT T7 Coupled Reticulocyte Lysate System (Promega, Madison, Wis.). In vitro translated proteins were combined at room temperature for 1 h, and reciprocal co-immunoprecipitation experiments were performed using mouse anti-MYC monoclonal or rabbit anti-HA polyclonal antibodies (Clontech).

### Example 13

Cell Culture, Plasmids and Transfection

CHO-K1 cells (American Type Culture Collection, Manassas, Va.) were cultured in Dulbecco's modified Eagle's medium/Ham's F-12 (DMEM/F-12) containing 10% fetal bovine serum (FBS) and grown to 90-95% confluence in 6 cm dishes. To express tagged proteins, mouse cDNAs were inserted into pCMV-Tag4A/FLAG-C and pCMV-Tag5A/MYC-C vectors (Stratagene, La Jolla, Calif.) and transiently transfected using LipofectAMINE 2000 (Invitrogen Life Technologies). Twenty-four hours after transfection, cells were harvested, processed in lysis buffer [50 mM TrisHCl, pH 7.4, 150 mM NaCl, 1 mM EDTA, 1% Triton X-100 and protease inhibitor cocktail (Sigma, St. Louis, Mo.)], and analyzed by immunoprecipitation and SDS-PAGE.

### Example 14

Cell Extract Immunoprecipitation and Western Blot Analysis

Immunoprecipitations were performed using the FLAG-Tagged Protein Immunoprecipitation kit (Sigma) as described by the manufacturer. The bound antibody was detected by the ECL detection kit (Amersham Pharmacia Biotech, Buckinghamshire, UK).

### Example 15

RFPL4 Protein was Expressed in Growing Oocytes and Early Embryos

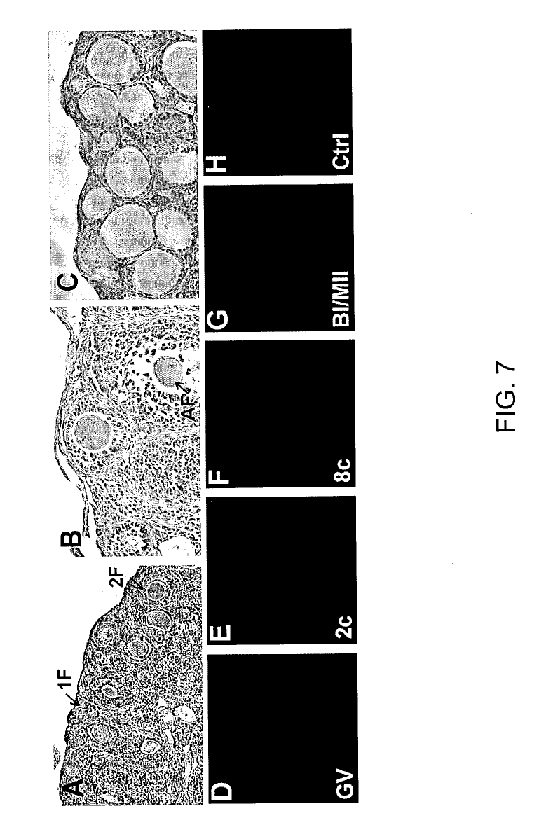

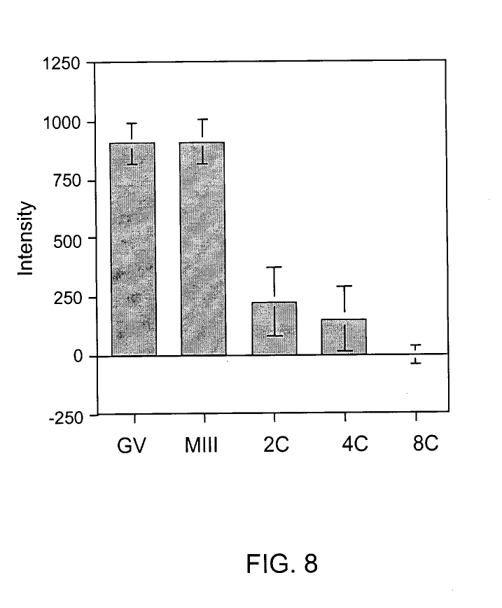

RFPL4 protein was located primarily in the cytoplasm of growing oocytes in both wild-type and Gdf9−/− mice, beginning at the primary follicle stage and extending through the preovulatory follicle stage (FIG. 7A-FIG. 7C). Deconvolution microscopy was used to assess RFPL4 protein expression in oocytes throughout meiotic maturation and in early preimplantation embryos. Fully grown oocytes were evaluated before the resumption of meiosis [germinal vesicle (GV) stage] and after ovulation and progression to metaphase II (FIG. 7H). In GV stage oocytes, RFPL4 protein was located predominantly in the cytoplasm and was relatively excluded from the nucleolus (FIG. 7E); after nuclear membrane breakdown, RFPL4 was detected throughout the oocyte (FIG. 7H). No expression was discemable in oocyte-associated granulosa cells. In addition, the RFPL4 protein was degraded in 2-cell stage and 8-cell stage embryos (FIG. 7F- FIG. 7G). Expression of RFPL4 was quantified by comparing immunofluorescent signal intensities in GV stage oocytes, metaphase II oocytes, 2-cell, 4-cell, and 8-cell embryos (FIG. 8). These data strongly suggested a specific role for RFPL4 in oogenesis and oocyte meiosis.

### Example 16

Yeast Two-Hybrid Screening of Mouse cDNA Libraries

The full-length open reading frame of mouse Rfpl4 (FIG. 9A) corresponding to amino acid residues 1-287 was subcloned into the pGBKT7 vector for expression as a GAL4 DNA binding fusion protein. Ovarian and oocyte cDNA libraries were subcloned into the pGADT7 vector to be expressed as transactivation domain fusion proteins.

In this yeast two-hybrid system, interactions between RFPL4 and proteins encoded by library cDNAs were expected to reconstitute transactivating complexes which bind to DNA and promote transcription of selectable markers. To identify RFPL4-interacting proteins, ˜1×10⁶ ovary cDNA transformants were screened by mating. A total of over 600 colonies grew on Leu-/Trp-/Ade-/His-/X-alpha-Gal selection plates and 27 of the isolated plasmids with inserts >500 bp were sequenced. Seven of these sequences were in-frame portions of the proteasome subunit β, type 1 (PSMB1) coding sequence (U60824).

Also, ˜1×10⁶ oocyte CDNA transformants were screened using the pGBKT7-RFPL4 bait. A total of over 200 colonies grew on the stringent selection plates, and 8 pGADT7 plasmids with large inserts were sequenced. One sequence corresponded to ubiquitin B cDNA (Ubb NM₁₃011664). One of the transformant cDNAs corresponded to PSMB 1. Other sequences identified in these screens are provided in Table 1.

TABLE 1
RFPL4-interactions identified in ovary cDNA library screen

| Putative interacting | | # of | | In vitro | |
|---|---|---|---|---|---|
| proteins | Accession # | clones | Cotransform | IP | CHO cell IP |
| Proteasome beta type 1 | U60824 | 7 (full- | + | + | + |
| subunit (PSMB1) | | length) | | | |
| Myosin light chain, | U04443 | 3 (full- | + | + | − |
| alkali, non-muscle | | length) | | | |
| (MYLN) | | | | | |
| Cytochrome C oxidase | AF378830 | 2 (full- | + | − | ND |
| subunit II (MT-CO2) | | length) | | | |
| Phosphoserine/threonine/ | U34973 | 1 | + | + | − |
| tyrosine interaction | | | | | |
| protein (STYX) | | | | | |
| Solute carrier family 25, | U27316 | 1 | + | − | ND |
| member 5 (SLC25A5) | | | | | |
| ATP synthase, H⁺ | BC010766 | 1 | − | − | ND |
| transporting, | | | | | |
| mitochondrial F₀ | | | | | |
| complex, subunit F | | | | | |
| (ATP5J) | | | | | |
| Lysosomal-associated | U34259 | 1 | − | − | ND |
| protein 4 (LAPTM4A) | | | | | |
| Microfibrillar-associated | NM_008546 | 1 | − | − | ND |
| protein 2 (MFAP2) | | | | | |

### Example 17

RFPL4-Interacting Proteins in Yeast

To test interactions between RFPL4 and HR6A, cyclin B1, PSMB1, or UbB in yeast, pGBKT7-RFPL4 and pGADT7 constructs were transformed and the growth with Leu-/Trp-/Ade-/His-/X-alpha-Gal selection was assessed. A fluorometric method for measuring yeast growth after cotransformation and mating was used to assess the strength of each interaction.

Briefly, after inoculation, the initial fluorescence was checked and the cells were allowed to grow for 40 hr, and then the fluorescence of an oxygen sensitive dye using the MATCHMAKER Biosensor kit (Clontech) was rechecked. Fluorescence was excited at 485 nm, and emission was read at 630 nm. To find the fold increase in fluorescence, an indication of yeast growth in selection media, the fluorescence intensity at time t=40 hr was divided by the initial intensity recorded at time t=0 hr (Fluorescence(t40)/Fluorescence(t0)). The values shown were the means of triplicate measurements of three independent transformants. Standard deviations were indicated by the error bars.

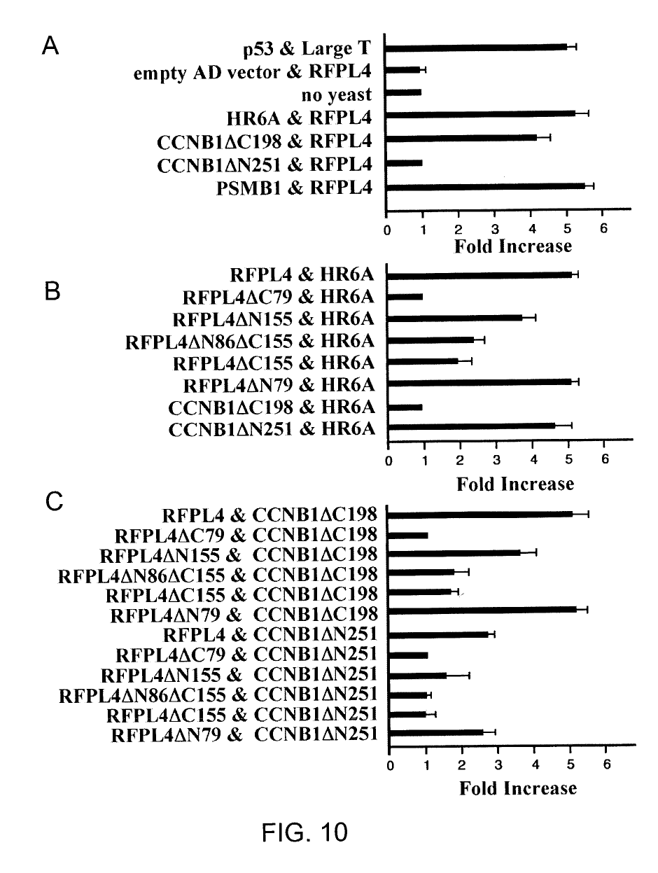

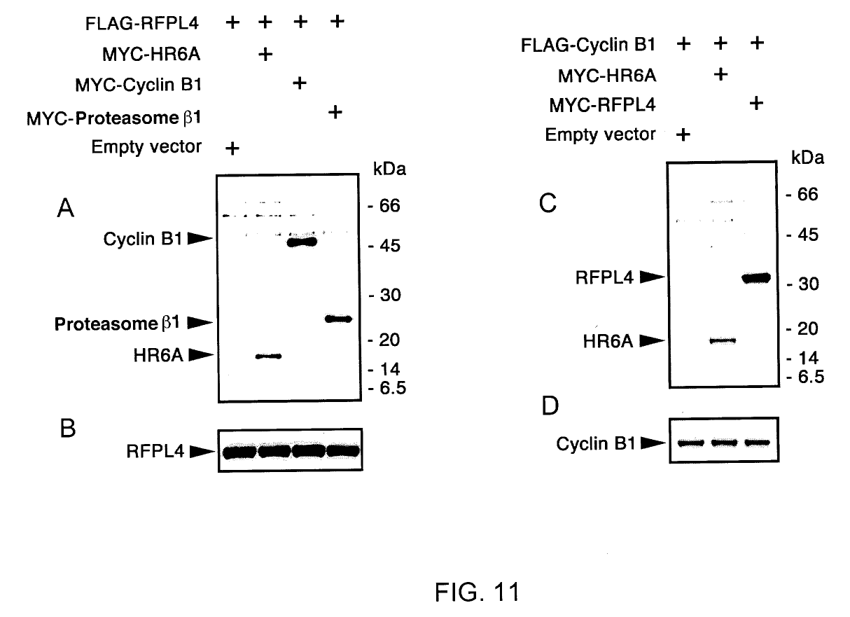

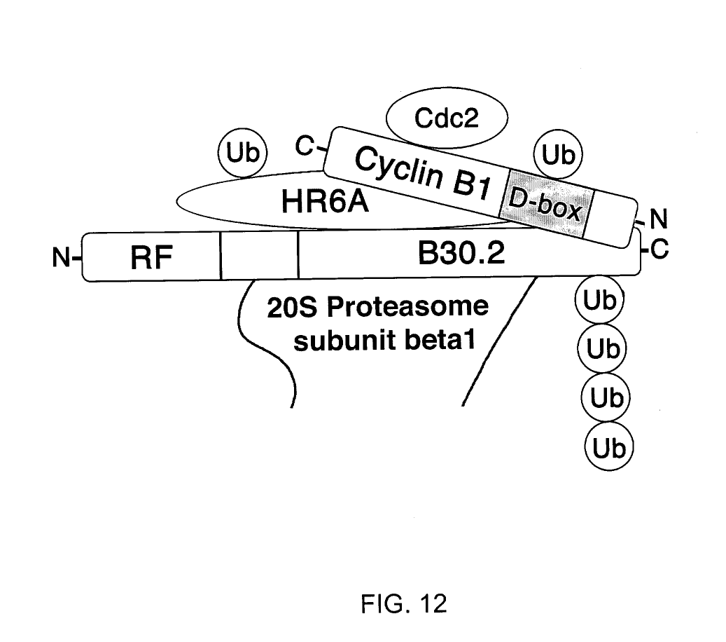

Cotransformants of pGBKT7-murine p53 and pGADT7-SV40 large T antigen were used as a positive control (>5-fold increase), and cotransformants of pGBKT7-RFPL4 and empty pGADT7 vector were used as a negative control (˜1-fold). Rapid growth was found in the cotransformants with pGBKT7-RFPL4 and pGADT7-HR6A, -N-terminal cyclin B1 (CCNB1ΔC 198), -PSMB1, or -UbB but not the cyclin B1 C-terminus (CCNB1ΔN251)(FIG. 10A). In contrast, cotransformant growth findings suggested that HR6A interacted strongly with the C-terminus of cyclin B1 (CCNB1Δ251), but not the N-terrninus of cyclin B1 (CCNB1ΔC198)(FIG. 10A). In addition, there was no growth of cotransformants with pGBKT7-RFPL4 and pGADT7-RFPL4, suggesting that RFPL4 did not interact with itself in yeast.

RFPL4 has a tripartite structure consisting of a cysteine-rich (C3YC4) RING finger-like region, a coiled-coiled motif, and a B30.2 domain, characteristics of RING-B30 family proteins (Rajkovic et al., 2002). To determine regions of RFPL4 that mediate its interactions, a series of truncation mutants (FIG. 9A) were engineered. The C-terminal B30.2 domain (amino acids 79-287; RFPL4ΔN79) proved both necessary and sufficient to recreate all of the interactions studied (FIG. 10B, FIG. 10C), which demonstrated that the RING finger-like region was dispensable for these strong interactions with HR6A and the N-terminus of cyclin B1. Deletion of the first 154 amino acids of RFPL4 (the RING finger-like region and a portion of the B30.2 domain; RFPL4ΔN155) weakened the interactions slightly with HR6A and CCNB1ΔC198 (FIG. 10B, FIG. 10C), but did not prevent the binding to UbB. Deletion of all of the B30.2 domain of RFPL4 (RFPL4ΔC79) or a C-terminal portion (RFPL4ΔN79ΔC155 and RFPL4ΔC155) abolished or significantly weakened, respectively, the interactions with HR6A (FIG. 10B). The RFPL4 B30.2 domain (RFPL4ΔN79 or RFPL4ΔN155) also interacted with the N-terminus of cyclin B1 (CCNB1ΔC198)(FIG. 10C). The RFPL4-cyclin B1 interaction data supported the findings that cyclin B1 ubiquitination depended upon on a destruction box (D-box) sequence located in the N-terrninus of cyclin B1 (Klotzbucher et al., 1996).

### Example 18

In Vitro Protein Interactions

To confirm the validity of the yeast two-hybrid results, co-immunoprecipitations of proteins expressed in vitro in a rabbit reticulocyte lysate system was performed. Epitope tagged bait and prey proteins were transcribed by T7 polymerase from pGBKT7 and pGADT7 templates without DNA binding or transactivation domains. As positive control vectors, pGBKT7-murine p53 (MYC-epitope tagged) and pGADT7-SV40 large T (HA-epitope tagged) were used. ³⁵S-methionine was included in translation mixtures to generate products detectable by autoradiography.

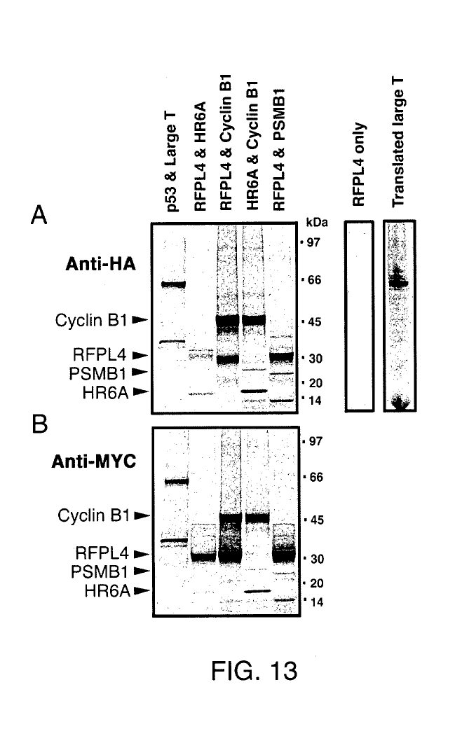

RFPL4 (32 kDa) interacted with HR6A (17 kDa) and full-length cyclin B1 (47 kDa) and HR6A bound full length cyclin B1 (FIG. 13A and FIG. 13B). Interactions between UbB (33 kDa) and RFPL4 (32 kDa) were not assessed as the two proteins could not be readily resolved by electrophoresis under these conditions. Other putative RFPL4 interactions identified in the yeast two hybrid screen were not verifiable by cotransformant assays and co-immunoprecipitation approaches (Table 1).

### Example 19

Protein Interactions in CHO Cells

To confirm that RFPL4 binds to HR6A, cyclin B1, and PSMB1 in mammalian cells, co-immunoprecipitation studies were performed using extracts of transiently transfected Chinese hamster ovary (CHO) cells. The interaction between HR6A (17 kDa) and cyclin B1 (47 kDa) was also studied by this approach. Anti-FLAG antibodies could co-immunoprecipitate FLAG-tagged, full-length RFPL4 bound to MYC-tagged HR6A, cyclin B1, or PSMB1 (25 kDa) from lysates of CHO cells cotransfected with these constructs (FIG. 11A). Likewise, the anti-FLAG antibodies co-immunoprecipitateed cyclin B1 and HR6A or cyclin B1 and RFPL4 (FIG. 11B). No deleterious effects were observed in any transiently transfected cells after 48 h of expression. Since the RFPL4 B30.2 domain interacted with cyclin B1 and HR6A in vitro, a truncated form of RFPL4 that lacked the RING finger-like motif (RFPL4ΔN79) was expressed, along with cyclin B1 or HR6A; however, binding was not demonstrated in CHO cells. In addition, the inventors examined MYC-tagged UbB co-expressed with FLAG-tagged RFPL4 in CHO cells, but no interaction was detected. These latter findings implied that these interactions were weak in CHO cells, possibly because of the presence of other factors that bind these proteins in cell culture.

### Example 20

Protein Interactions

Cell-free transcription/translation of Rfpl4 and Zar1 cDNAs, followed by co-immunoprecipitation and SDS-PAGE were performed as described in Example 14.

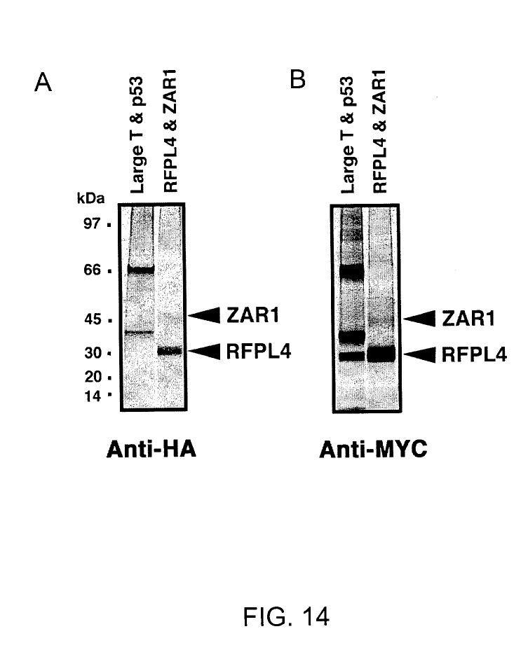

Autoradiograph of [³⁵S]Met-labeled proteins from cell-free in vitro transcription/translation and co-immunoprecipitation by anti-HA polyclonal antibody (FIG. 14A) or anti-MYC monoclonal antibody (FIG. 14B). The position of molecular mass standards in kDa was shown at left. The pGBKT7-murine p53 MYC-tagged (p53) and pGADT7-SV40 large T-antigen HA-tagged (Large T) were used for positive control. This data illustrated that the HA-tagged ZAR1 binds to the MYC-tagged RFPL4.

### Example 21

Generate an Rfpl4 Null Allele and Produce Rfpl4 Knockout Mice

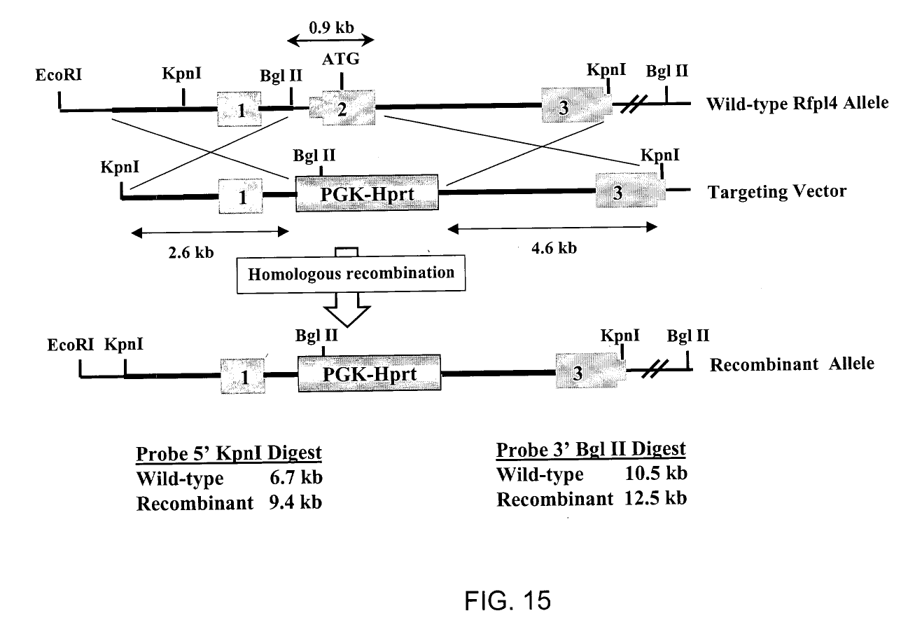

Rfpl4 is a 3 exon gene. About 20 kb of the mouse Rfpl4 genomic locus was isolated. An Rfpl4 targeting vector is being designed (FIG. 15) to delete exons 1 and 2 which contain the start of transcription (exon 1), the start of translation (exon 2), most of the protein coding domain (exon 2), and the putative RING finger-like domain (exon 2). The next coding methionine is at amino acid 222, near the C-terminus of the protein. The mutated locus should yield no Rfpl4 mRNA or protein and therefore will be a null allele. This targeting vector is electroporated into hprt-deficient ES cells, Rfpl4 mutant ES cells selected, clones injected into blastocysts, chimeras produced, heterozygotes generated, and Rfpl4 null mice (Rfpl4) produced from intercrosses of heterozygotes. The Rfpl4 null mice are viable given that Rfpl4 expression is limited to the ovaries and testes.

### Example 22

Analyze the Reproductive Performance of Rfpl4 Knockout Mice

To address the reproductive performance of Rfpl4 knockout mice, homozygous female mice are bred to wild-type males and homozygous males are bred to wild-type females for one year.

It is envisioned that Rfpl4 null mice of both sexes are infertile due to germ cell defects. Thus, it is necessary to analyze the morphological and histological appearance of the ovaries and testes of knockout and control adult and adolescent mice. For the testis analysis, morphological analysis includes weighing the testes at 3 weeks, 6 weeks, and 12 weeks (Kumar et al., 1997; Matzuk et al., 1992). If morphological or histological analyses detect testicular defects, stereological analysis of the various cells of the testis are performed with FSHβ, ActRII, or double mutant mice.

For the ovaries, histological analysis is performed on 12-week old knockout and control mice to determine whether all follicle types and corpora lutea are present. Superovulation experiments are performed to quantitate the numbers of oocytes released, their ability to complete meiosis, the ability of the eggs to be fertilized in vivo and in vitro, and the capability of fertilized eggs to develop to the blastocyst stage.

In females, it is envisioned that absence of RFPL4 leads to a block at the metaphase I-anaphase I transition and thereby causes female fertility. Experiments are performed to confirm that ovulated oocytes do not have a first polar body and instead show an arrest at metaphase I-anaphase I (Viveriros et al., 2001). Thus, these arrested oocytes are not competent to develop to term. However, oocytes arrested at metaphase I are competent to undergo fertilization and even development to the blastocyst stage. Nevertheless, fertilization of oocytes at metaphase I results in the production of triploid embryos, which cannot develop to term (Eppig et al., 1994).

If the progression of meiosis is arrested at metaphase I-anaphase I, then it is envisioned that RFPL4 regulates cyclin B1 degradation. Thus, to confirm this aspect, experiments include testing the MPF activity (i.e., H1 kinase activity) of wild-type and Rfpl4 knockout oocytes cultured in vitro. It is contemplated that MPF activity increases after 12 and 24 hours of culture. In addition, this oocyte defect does not affect the architecture of the ovary nor perturb the hormonal milieu. Then contraceptives designed to block RFPL4 function will not affect the pool of oocytes, the processes of folliculogenesis or luteinization, normal menstrual cycling, and estrogen and progesterone levels, and therefore not require co-administration of estrogen supplements to prevent osteopenic changes.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Albrecht, U. et al., (1997) Visualization of gene expression patterns by in situ hybridization. In Daston, G. P. (ed.), Molecular and Cellular Methods in Developmental Toxicology. CRC Press, Boca Raton,, pp. 23-48.

Arcone, et al., (1988) Nucl. Acids Res., 16(8): 3195-3207.

Aruffo et al. (1987) EMBO J. 6: 3313.

Baichwal and Sugden, In: Gene Transfer, pp. 117-148, 1986.

Barany and Merrifield, (1979) The Peptides, pp. 1-284.

Bartel, P. L. and S. Fields In: THE YEAST TWO-HYBRID SYSTEM. Oxford University Press, 1997.

Benvenisty and Neshif, (1986) Proc. Nat'l Acad Sci. USA, 83:9551-9555.

Berzal-Herranz et al, (1992) Genes Dev. 6(1):129-34.

Bohrnann et al., (1989) Cell. November 17;59(4):709-17.

Brinster et al., (1985) Proc. Nat'l Acad. Sci. USA, 82: 4438-4442.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Carter and Flotte, (1995) Ann. N.Y. Acad. Sci., 770:79-90.

Cech et al., (1981) Cell. December ;27(3 Pt 2):487-96.

Chatterjee, et al, (1995) Ann. N. Y Acad. Sci., 770:79-90.

Chen and Okayama (1987) Mol. Cell Biol., 7:2745-2752.

Chowrira et al, (1994) J Biol Chem. 269(41):25856-64.

Chowrira et al., (1993) J Biol Chem. 268(26):19458-62.

Coffin, (1990) In: Virology, ed., New York: Raven Press, pp. 1437-1500.

Coupar et al., (1988) Gene, 68:1-10.

Courey et al., (1988) Cell. 55(5):887-98.

Davey et al., EPO No. 329 822.

Dong, J. et al., (1996) Nature 383, 531-5.

Dubensky et al., (1984) Proc. Nat'l Acad Sci. USA, 81:7529-7533.

Elvin, J. A. et al., (1999) Mol Endocrinol 13, 1018-34.

Eppig et al., (1994) Human Reprod. 9, 1136-42.

Fashena et al., (2000) Methods Enzymol. 328:14-26.

Fechheimer et al., (1987) Proc. Nat'l Acad Sci. USA, 84:8463-8467.

Ferrari et al., (1996) J. Virol., 70:3227-3234.

Fisher et al., (1996) J. Virol., 70:520-532.

Flotte et al., (1993) PROC. Nat'l Acad. Sci. USA, 90:10613-10617.

Fodor et al., (1991) Science, 251:767-773.

Forster and Symons, (1987) Cell, 49:211-220.

Fraley et al. (1979) Proc. Nat'l Acad. Sci USA, 76:3348-3352.

Freemont, P. S. (2000) Curr Biol 10, R84-7.

Friedman et al., (1990) Genes Dev. Aug;4(8):1416-26.

Gefter et al., (1977) Somatic Cell Genet. 3:231-236.

Gerster et al., (1990) EMBO J. (5):1635-43.

Ghosh and Bachhawat, (1991) In: Liver Diseases, Targeted Diagnosis and Therapy Using Specific Receptors and Ligands. Wu et al., pp. 87-104.

Giet et al., (2001) J. Cell Biol. 152(4):669-82.

Gingeras et al., PCT Application WO 88/10315.

Goding, (1986) In: Monoclonal Antibodies: Principles and Practice, 71-74.

Goodman et al., (1994) Blood, 84:1492-1500.

Gopal, (1985) Mol. Cell Biol., 5:1188-1190.

Gossen et al., (1995) Science, 268:1766-1769.

Gossen and Bujard (1992) Proc. Nat'l Acad. Sci. USA, 89:5547-5551.

Graham and van der Eb, (1973) Virology, 52:456-467.

Hacia et al., (1996) Nature Genetics, 14:441-447.

Hammond et al., (2001) Nat Rev Genet. 2(2):110-9.

Harland and Weintraub, (1985) J. Cell Biol., 101:1094-1099.

Haseloff and Gerlach (1988) Nature. 334(6183):585-91.

Hay (1984) J. Mol. Biol., 175:493-510.

Hearing and Shenk, (1983) J. Mol. Biol. 167:809-822.

Hearing et al., (1987) J. Virol., 67:2555-2558.

Henry, J. et al., (1997) Biochem Biophys Res Commun 235, 162-5.

Henry, J. et al., (1998) Mol Biol Evol 15, 1696-705.

Hope et al., (1987) EMBO J. Sep;6(9):2781-4.

Hu et al., (2000) Methods. 20(1):80-94.

James et al., (1996) Genetics. 144(4):1425-36.

Joazeiro, C. A. and Weissman, A. M. (2000) Cell 102, 549-52.

Johnson et al., (1993) Peptide Turn Mimetics” In: Biotechnology And Pharmacy.

Joyce Nature, (1989) 338:217-244.

Kageyama et al., (1987) J. Biol. Chem., 262(5):2345-2351.

Kaneda et al., (1989) Science, 243:375-378.

Kaplitt et al., (1994) Nat'l Genet., 8:148-153.

Kato et al, (1991) J. Biol. Chem., 266:3361-3364.

Kim and Cook, (1987) Proc. Nat'l Acad. Sci. USA, 84:8788-8792.

Klein et aL,(1987) Nature, 327:70-73.

Ko, M. S. et al., (2000) Development. 127, 1737-49.

Koeberl et al., (1997) Proc. Nat'l Acad. Sci. USA, 94:1426-1431.

Kohler and Milstein, (1976) Eur. J. Immunol., 6:511-519.

Kumar, T. R. et al., (1997) Nature Genetics, 15, 201-204.

Legrain, P. et al., (2001) Trends Genet. 17, 346-52.

Levrero et al., (1991) Gene, 10 1: 195-202.

Lieber et al., (1995) Mol Cell Biol. 15(1):540-51.

Lira SA et al., Proc. Nat'l Acad. Sci. USA (1990) September;87(18):7215-9.

Ma et al., (1987) 50:137-42.

Mann etal., (1983) Cell, 33:153-159.

Martin et al., (1990) Genes Dev. (11):1886-98.

Matzuk et al., (1992) Nature. 360: 313-319.

McCown et al., (1996) Brain Res., 713:99-107.

Mermod et al., (1989) Cell. 58(4):741-53.

Merrifield, (1986) Science, 232: 341-347.

Michel and Westhof (1990) J. Mol. Biol, 216:585-610.

Muller-Immergluck et al., (1990) EMBO J 9(5):1625-34.

Nakamura et al., (1987) In: Handbook of Experimental Immunology (4th Ed.).

Nicolau and Sene (1982) Biochim. Biophys. Acta, 721:185-190.

Nicolau et al., (1987) Methods Enzymol., 149:157-176.

Oliviero (1987) et al., EMBO J., 6(7):1905-1912.

Paskind et al., (1975) Virology, 67:242-248.

Pease et al.,(1994) Proc. Nat'l Acad. Sci. USA, 91:5022-5026.

Perriman et al., (1992) Gene. 113(2): 157-63.

Perrotta and Been (1992) Biochemistry. 31(1):16-21.

Peschon J J et al., Proc. Nat'l Acad Sci. USA (1987) August ;84(15):5316-9.

Ping et al., (1996) Microcirculation, 3:225-228.

Poli and Cortese, (1989) Proc. Nat'l Acad. Sci. USA, 86:8202-8206.

Potter et al., (1984) Proc. Nat'l Acad. Sci. USA, 81:7161-7165.

Prowse and Baumann (1988) Mol Cell Biol, 8(1):42-51.

Quaderi, N. A. et al., (1997) Nat Genet 17, 285-91.

Radler et al., (1997) Science, 275:810-814.

Rajkovic, A. et al., (2001) Fertil Steril 76, 550-4.

Reinhold-Hurek and Shub, (1992) Nature, 357:173-176.

Renan (1990) Radiother. Oncol., 19:197-218.

Ridgeway, (1988) In: Vectors: A survey of molecular cloning vectors and their uses, pp. 467-492.

Rippe et al., (1990) Mol. Cell Biol., 10:689-695.

Ron, et al., (1991) Mol. Cell. Biol., 2887-2895.

Roux et al., (1989) Proc. Nat'l Acad. Sci. USA, 86:9079-9083.

Sadowski et al., (1988) Nature. 335(6190):563-4.

Sambrook et al., (1989) MOLECULAR CLONING: A LABORATORY MANUAL, 2nd Ed., Cold Spring Harbor Laboratory Press, Cold Spring Harbor, N.Y.

Samulski et al., (1987) J. Virol., 61(10):3096-3101.

Sarver et al.,(1990) Science, 247:1222-1225.

Scanlon et al., (1991) Proc. Nat'l Acad. Sci. USA, 88:10591-10595.

Seed et al., (1987) Proc. Nat'l Acad. Sci. USA, 84: 3365.

Scroussi, E. et al., (1999) Genome Res 9, 803-14.

Seto, M. H. et al., (1999) Proteins 35, 235-49.

Shoemaker et al., (1996) Nature Genetics 14:450-456.

Sioud et al., (1992) J. Mol Biol 223(4):831-5.

Stein P, et al., Biochem Biophys Res Commun. 2002 291(5):1119-22.

Stewart and Young, (1984) Solid Phase Peptide Synthesis, 2d. ed., Pierce Chemical Co.

Svoboda P, et al., Biochem Biophys Res Commun. 2001 287(5):1099-104.

Svoboda P, et al., Development. 2000;127(19):4147-56.

Symons, Annu Rev Biochem. (1992) ;61:641-71.

Tam et al., (1983) J. Am. Chem. Soc., 105:6442.

Temin, (1986) In: Gene Transfer, Kucherlapati (ed.), New York: Plenum Press, pp. 149-188.

Thai, T. H. et al., (1998) Hum Mol Genet 7, 195-202.

Thompson et al., (1995) Nucleic Acids Res. 23(12):2259-68.

Tibbetts (1977) Cell, 12:243-249.

Tur-Kaspa et al., (1986) Mol. Cell Biol., 6:716-718.

Vidal, M. et al., (1999) Nucleic Acids Res. 27, 919-29.

Viveiros, M. M. et al., (2001) Dev. Biol. 235, 330-42.

Watt et al., (1986) Proc. Nat'l Acad. Sci., 83(2): 3166-3170.

Wilson et al., (1990) Mol. Cell. Biol., 6181-6191.

Wong et al., (1980) Gene, 10:87-94.

Wong et al.,(1985) Science, 228: 810-815.

Wu and Wu, (1987) J. Biol. Chem., 262:4429-4432.

Wu and Wu, (1988) Biochem., 27:887-892.

Wu, L. C., et al., (1996) Nat Genet 14, 430-40.

Xiao et al., (1996) J. Virol., 70:8098-8108.

Yang et al., (1990) Proc. Nat'l Acad. Sci. USA, 87:9568-9572.

Yuan and Altman (1994) Science. 263(5151):1269-73.

Yuan et al., (1992) Proc. Nat'l Acad. Sci. USA 89(17):8006-10.

Zechner et al., (1988) Mol. Cell. Biol., 2394-2401.

Zhang L P et al., Biol Reprod. (1999) Jun;60(6):1329-37.

Zhang, Y. et al., (2000) Proc. Nat'l Acad. Sci. USA 24, 13354-9.

Although the present invention and its advantages have been described in detail, it should be understood that various changes, substitutions and alterations can be made herein without departing from the spirit and scope of the invention as defined by the appended sentences. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended sentences are intended to include within their scope such processes, machines, manufacture, compositions of matter, means, methods, or steps.

We claim:

1. An isolated polynucleotide sequence encoding a polypeptide comprising an amino acid sequence of SEQ.ID.NO: 3 or complement thereof.

2. An isolated polynucleotide sequence comprising a nucleic acid sequence of SEQ.ID.NO: 1.

3. An isolated polynucleotide sequence encoding a polypeptide comprising an amino acid sequence of SEQ.ID.NO: 4 or complement thereof.

4. An isolated polynucleotide sequence comprising a nucleic acid sequence of SEQ.ID.NO: 2.

5. An isolated polynucleotide sequence encoding a polypeptide comprising an amino acid sequence of SEQ.ID.NO: 5 or complement thereof.

6. An isolated polynucleotide sequence comprising a nucleic acid sequence of SEQ.ID.NO: 6.

7. An isolated polypeptide comprising an amino acid sequence of SEQ.ID.NO: 3.

8. An isolated polypeptide comprising an amino acid sequence of SEQ.ID.NO: 4.

9. An isolated polypeptide comprising an amino acid sequence of SEQ.ID.NO: 5.

10. A monoclonal antibody that binds immunologically to a polypeptide comprising SEQ.ID.NO: 3, or an antigenic fragment thereof

11. A monoclonal antibody that binds immunologically to a polypeptide comprising SEQ.ID.NO: 4, or an antigenic fragment thereof.

12. A monoclonal antibody that binds immunologically to a polypeptide comprising SEQ.ID.NO: 5, or an antigenic fragment thereof.

13. A polyclonal antisera, antibodies of which bind immunologically to a polypeptide comprising SEQ.ID.NO: 3, or an antigenic fragment thereof

14. A polyclonal antisera, antibodies of which bind immunologically to a polypeptide comprising SEQ.ID.NO: 4, or an antigenic fragment thereof.

15. An expression vector comprising a polynucleotide sequence encoding a polypeptide having an amino acid sequence of SEQ.ID.NO: 3, wherein said polynucleotide is under control of a promoter operable in cells.

16. An expression vector comprising a polynucleotide sequence encoding a polypeptide having an amino acid sequence of SEQ.ID.NO: 4, wherein said polynucleotide is under control of a promoter operable in cells.

17. The expression vector of claim 15, wherein said polynucleotide sequence comprises SEQ.ID.NO: 1.

18. The expression vector of claim 16, wherein said polynucleotide sequence comprises SEQ.ID.NO: 2.

19. A host cell transformed with the expression vector of claim 15.

20. A host cell transformed with the expression vector of claim 16.

21. A method for producing a polypeptide comprising the steps of:
culturing a host cell according to claim 19 under conditions suitable for the expression of said polypeptide; and
recovering said polypeptide from the host cell culture.

22. A method for producing a polypeptide comprising the steps of:
culturing a host cell according to claim 20 under conditions suitable for the expression of said polypeptide; and
recovering said polypeptide from the host cell culture.

23. A pharmaceutical composition comprising a modulator of RFPL4 expression dispersed in a pharmaceutically acceptable carrier.

24. The composition of claim 23, wherein the modulator suppresses transcription of an RFPL4 gene.

25. The composition of claim 23, wherein the modulator suppresses translation of an RFPL4 transcript.

26. The composition of claim 23, wherein the modulator alters RNA stability by increasing RNA degradation.

27. The composition of claim 23, wherein the modulator enhances transcription of an RFPL4 gene.

28. The composition of claim 23, wherein the modulator enhances translation of an RFPL4 transcript.

29. The composition of claim 23, wherein the modulator alters RNA stability by decreasing RNA degradation.

30. The composition of claim 23, wherein the modulator is a polypeptide.

31. The composition of claim 23, wherein the modulator is a polynucleotide sequence.

32. The composition of claim 31, wherein the polynucleotide sequence is DNA or RNA.

33. The composition of claim 32 further comprising an expression vector, wherein the expression vector comprises a promoter and the polynucleotide sequence, operatively linked.

34. A pharmaceutical composition comprising a modulator of RFPL4 activity dispersed in a pharmaceutically acceptable carrier.

35. The composition of claim 34, wherein the composition inhibits RFPL4 activity.

36. The composition of claim 34, wherein the composition stimulates RFPL4 activity.

37. A method of identifying compounds that modulate the activity of RFPL4 comprising the steps of:
obtaining an isolated RFPL4 polypeptide or functional equivalent thereof;
admixing the RFPL4 polypeptide or functional equivalent thereof with a candidate compound;
and measuring an effect of said candidate compound on the activity of RFPL4.

38. The method of claim 37, wherein the effect is a decrease in protein degradation.

39. The method of claim 37, wherein the effect is a increase in protein degradation.

40. A method of screening for a modulator of RFPL4 activity comprising the steps of:
providing a cell expressing an RFPL4 polypeptide
contacting said cell with a candidate compound;
measuring RFPL4 expression; and
comparing said RFPL4 expression in the presence of said candidate compound with the expression of RFPL4 expression in the absence of said candidate compound;
wherein a difference in the expression of RFPL4 in the presence of said candidate compound, as compared with the expression of RFPL4 in the absence of said candidate compound, identifies said candidate modulator as a modulator of RFPL4 expression.

41. A method of producing a modulator of RFPL4 activity comprising the steps of:
providing a cell expressing an RFPL4 polypeptide
contacting said cell with a candidate compound;
measuring RFPL4 expression;
comparing said RFPL4 expression in the presence of said candidate compound with the expression of RFPL4 expression in the absence of said candidate compound; wherein a difference in the expression of RFPL4 in the presence of said candidate compound, as compared with the expression of RFPL4 in the absence of said candidate compound, identifies said candidate compound as a modulator of RFPL4 expression; and
producing the modulator.

42. A method of modulating protein degradation in a germ cell or early embryo of an animal comprising the step of administering to the animal an inhibitor of RFPL4 activity.

43. The method of claim 42, wherein said germ cell is an oocyte or egg.

44. The method of claim 42, wherein said germ cell is spermatogonium, spermatocyte, spermatid or spermatazoon.

45. The method of claim 42, wherein the inhibitor suppresses transcription of an RFPL4 gene.

46. The method of claim 42, wherein the inhibitor suppresses translation of an RFPL4 transcript.

47. The method of claim 42, wherein the inhibitor alters RNA stability by increasing RNA degradation.

48. The method of claim 42, wherein the inhibitor is a polypeptide.

49. The method of claim 42, wherein the inhibitor is a polynucleotide sequence.

50. The method of claim 49, wherein the polynucleotide sequence is DNA or RNA.

51. The method of claim 49 further comprising an expression vector, wherein the expression vector comprises a promoter and the polynucleotide sequence, operatively linked.

52. The method of claim 51, wherein the vector is a bacterial, viral or mammalian vector.

53. The method of claim 50, wherein the RNA is an antisense RFPL4 RNA.

54. The method of claim 50, wherein the RNA is an RNA interference of RFPL4 RNA.

55. A method of contraception comprising administering to an animal an effective amount of an inhibitor of RFPL4 activity dispersed in a pharmacologically acceptable carrier, wherein said amount is capable of decreasing conception.

56. The method of claim 55, wherein the animal is female.

57. The method of claim 55, wherein the animal is male.

58. The method of claim 55, wherein the inhibitor suppresses transcription of an RFPL4 gene.

59. The method of claim 55, wherein the inhibitor suppresses translation of an RFPL4 transcript.

60. The method of claim 55, wherein the inhibitor alters RNA stability by increasing RNA degradation.

61. The method of claim 55, wherein the inhibitor is a polypeptide.

62. The method of claim 55, wherein the inhibitor is a polynucleotide sequence.

63. The method of claim 62, wherein the polynucleotide sequence is DNA or RNA.

64. The method of claim 62 further comprising an expression vector, wherein the expression vector comprises a promoter and the polynucleotide sequence, operatively linked.

65. The method of claim 64, wherein the wherein the vector is a bacterial, viral or mammalian vector.

66. The method of claim 63, wherein the RNA is an antisense RFPL4 RNA.

67. The method of claim 63, wherein the RNA is an RNA interference of RFPL4 RNA.

68. A method of contraception comprising administering to an animal an effective amount of a stimulator of RFPL4 activity dispersed in a pharmacologically acceptable carrier, wherein said amount is capable of decreasing conception.

69. The method of claim 68, wherein the animal is female.

70. The method of claim 68, wherein the animal is male.

71. The method of claim 68, wherein the stimulator enhances transcription of an RFPL4 gene.

72. The method of claim 68, wherein the stimulator enhances translation of an RFPL4 transcript.

73. The method of claim 68, wherein the stimulator alters RNA stability by decreasing RNA degradation.

74. The method of claim 68, wherein the stimulator is a polypeptide.

75. The method of claim 68, wherein the stimulator is a polynucleotide sequence.

76. The method of claim 75, wherein the polynucleotide sequence is DNA or RNA.

77. The method of claim 75 further comprising an expression vector, wherein the expression vector comprises a promoter and the polynucleotide sequence, operatively linked.

78. The method of claim 77, wherein the wherein the vector is a bacterial, viral or mammalian vector.

79. The method of claim 76, wherein the RNA is an antisense RFPL4 RNA.

80. The method of claim 76, wherein the RNA is an RNA interference of RFPL4 RNA.

81. A method of modulating protein degradation in a germ cell or early embryo of an animal comprising the step of administering to the animal a stimulator of RFPL4 activity.

82. The method of claim 81, wherein said germ cell is an oocyte or egg.

83. The method of claim 81, wherein said germ cell is spermatogonium, spermatocyte, spermatid or spermatazoon.

84. The method of claim 81, wherein the stimulator enhances transcription of an RFPL4 gene.

85. The method of claim 81, wherein the stimulator enhances translation of an RFPL4 transcript.

86. The method of claim 81, wherein the stimulator alters RNA stability by decreasing RNA degradation.

87. The method of claim 81, wherein the stimulator is a polypeptide.

88. The method of claim 81, wherein the stimulator is a polynucleotide sequence.

89. The method of claim 88, wherein the polynucleotide sequence is DNA or RNA.

90. The method of claim 88 further comprising an expression vector, wherein the expression vector comprises a promoter and the polynucleotide sequence, operatively linked.

91. The method of claim 90, wherein the wherein the vector is a bacterial, viral or mammalian vector.

92. The method of claim 89, wherein the RNA is an antisense RFPL4 RNA.

93. The method of claim 89, wherein the RNA is an RNA interference of RFPL4 RNA.

94. A method of enhancing fertility comprising administering to an animal an effective amount of an inhibitor of RFPL4 activity dispersed in a pharmacologically acceptable carrier, wherein said amount is capable of decreasing conception.

95. The method of claim 94, wherein the animal is male.

96. The method of claim 94, wherein the animal is female.

97. The method of claim 94, wherein the inhibitor suppresses transcription of an RFPL4 gene.

98. The method of claim 94, wherein the inhibitor suppresses translation of an RFPL4 transcript.

99. The method of claim 94, wherein the inhibitor alters RNA stability by increasing RNA degradation.

100. The method of claim 94, wherein the inhibitor is a polypeptide.

101. The method of claim 94, wherein the inhibitor is a polynucleotide sequence.

102. The method of claim 101, wherein the polynucleotide sequence is DNA or RNA.

103. The method of claim 101 further comprising an expression vector, wherein the expression vector comprises a promoter and the polynucleotide sequence, operatively linked.

104. The method of claim 103, wherein the wherein the vector is a bacterial, viral or mammalian vector.

105. The method of claim 102, wherein the RNA is an antisense RFPL4 RNA.

106. The method of claim 102, wherein the RNA is an RNA interference of RFPL4 RNA.

107. A method of enhancing fertility comprising administering to an animal an effective amount of a stimulator of RFPL4 activity dispersed in a pharmacologically acceptable carrier, wherein said amount is capable of decreasing conception.

108. The method of claim 107, wherein the animal is female.

109. The method of claim 107, wherein the animal is male.

110. The method of claim 107, wherein the stimulator enhances transcription of an RFPL4 gene.

111. The method of claim 107, wherein the stimulator enhances translation of an RFPL4 transcript.

112. The method of claim 107, wherein the stimulator alters RNA stability by decreasing RNA degradation.

113. The method of claim 107, wherein the stimulator is a polypeptide.

114. The method of claim 107, wherein the stimulator is a polynucleotide sequence.

115. The method of claim 114, wherein the polynucleotide sequence is DNA or RNA.

116. The method of claim 114 further comprising an expression vector, wherein the expression vector comprises a promoter and the polynucleotide sequence, operatively linked.

117. The method of claim 116, wherein the wherein the vector is a bacterial, viral or mammalian vector.

118. The method of claim 115, wherein the RNA is an antisense RFPL4 RNA.

119. The method of claim 115, wherein the RNA is an RNA interference of RFPL4 RNA.

120. A method of diagnosing infertility comprising identifying a mutation in an RFPL4 polypeptide or polynucleotide.

121. The method of claim 120, wherein said method comprises identifying a mutation in an RFPL4 polypeptide.

122. The method of claim 121, wherein said method comprises identifying a mutation in an RFPL4 polynucleotide.

123. The method of claim 122, wherein said polynucleotide is RFPL4 mRNA.

124. The method of claim 122, wherein said polynucleotide is RFPL4 genomic DNA.

125. The method of claim 122, wherein said method comprises amplification of said polynucleotide.

126. The method of claim 122, wherein said method comprises hybridization of said polynucleotide to a labeled polynucleotide.

127. The method of claim 122, wherein said method comprises sequencing of an RFPL4 polynucleotide.
